(19) United States
(12) Patent Application Publication
(10) Pub. No.: US 2019/0374601 A1
(43) Pub. Date: Dec. 12, 2019

(54) METHODS FOR TREATING FUNGAL INFECTIONS

(71) Applicant: Cidara Therapeutics, Inc., San Diego, CA (US)

(72) Inventors: Kenneth BARTIZAL, La Jolla, CA (US); Paul DARUWALA, Del Mar, CA (US)

(21) Appl. No.: 16/482,043

(22) PCT Filed: Jan. 31, 2018
(86) PCT No.: PCT/US2018/016236

§ 371 Date: Jul. 30, 2019

Related U.S. Application Data
(60) Provisional application No. 62/452,643, filed on Jan. 31, 2017.

(51) Int. Cl. A61K 38/12; A61K 9/00; A61P 31/10

(52) U.S. Cl. CPC A61K 38/12; A61K 31/137; A61P 31/10; A61K 9/0019

(57) ABSTRACT

The invention relates to methods of treating dermatophytosis (e.g., onychomycosis) in a subject by subcutaneously administering to the subject CD101, in salt or neutral form. In some embodiments, CD101 in salt or neutral form is administered in combination with at least one antifungal agent.

### BACKGROUND

Dermatophytosis, including onychomycosis, causes significant discomfort and affects patients' quality-of-life. Currently available antifungal preparations, e.g., echinochandins, do not provide adequate cure for dermatophytosis. The three echinocandins approved by the Food and Drug Administration (FDA) for the treatment of invasive fungal infections (caspofungin, anidulafungin, and micafungin) are available only in intravenous formulations and have not been used for the treatment of dermatophytosis. There is a need in the art for improved methods of treatment for dermatophytosis.

### SUMMARY

The invention relates to methods of treating dermatophytosis (e.g., dermatophytic onychomycosis) or non-dermatophytic onychomycosis in a subject (e.g., a human) by administering (e.g., subcutaneously administering to the subject a salt of CD101, or a neutral form thereof. A salt of CD101, or a neutral form thereof, displays long-acting pharmacokinetics with a long half-life, slow clearance, and strong activities against dermatophytes (e.g., Trichophyton, Microsporum, Epidermophyton) and fungi in other genera (e.g., Candida). The stability of CD101 in salt or neutral form especially enables non-intravenous formulations of CD101, e.g., subcutaneous formulations.

In a first aspect, the invention features a method of treating onychomycosis in a subject. The method includes subcutaneously administering to the subject doses of about 25 mg to about 600 mg of CD101,

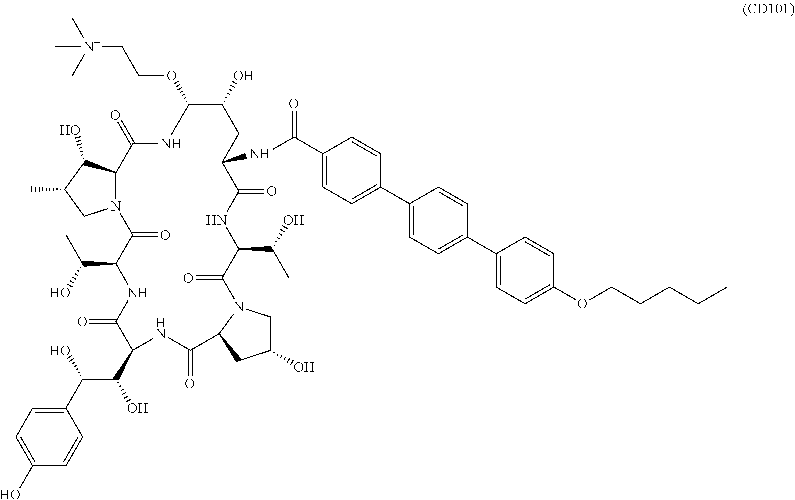 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1]

in salt or neutral form, wherein the CD101 in salt or neutral form is administered in one or more doses to the subject over a period of 4 to 52 weeks and wherein no more than one dose is administered per week.

In some embodiments, one dose of CD101 in salt or neutral form is administered once every 4 weeks over a period of 4 to 52 weeks. In some embodiments, one dose of CD101 in salt or neutral form is administered once every 3 weeks over a period of 6 to 51 weeks. In some embodiments, one dose of CD101 in salt or neutral form is administered once every 2 weeks over a period of 4 to 52 weeks. In some embodiments, one dose of CD101 in salt or neutral form is administered once a weeks over a period of 4 to 52 weeks.

In some embodiments, one or more doses of CD101 is administered over a period of 12 to 24 weeks. In some embodiments, 3 to 12 doses of CD101 is administered over a period of 12 weeks. In some embodiments, 4 to 16 doses of CD101 is administered over a period of 16 weeks. In some embodiments, 5 to 20 doses of CD101 is administered over a period of 20 weeks. In some embodiments, 6 to 24 doses of CD101 is administered over a period of 24 weeks.

In another aspect, the invention features a method of preventing or reducing the likelihood of onychomycosis in a subject by subcutaneously administering to the subject CD101 in salt or neutral form.

In some embodiments of this aspect, the subject is in a population with high prevalence of onychomycosis (e.g., a population of soldiers, long-distance runners, or miners).

In some embodiments of the methods described herein, the onychomycosis is a dermatophytic onychomycosis. In some embodiments, the dermatophytic onychomycosis is caused by a fungus in the genus Trichophyton, Microsporum, or Epidermophyton.

In some embodiments, the fungus is in the genus Trichophyton (e.g., Trichophyton rubrum, T. mentagrophytes, T. interdigitale, T. violaceum, T. tonsurans, T. soudanense, T. concentricum, T. megnini, T. schoenleinii, T. yaoundei, T. verrucosum, T. simii, T. redellii, T. equinum, T. ajelloi, T. vanbreuseghemii, T. terrestre, T. phaseoliforme, T. flavescens, T. gloriae, or T. onychocola). In some embodiments, the fungus is T. rubrum or T. mentagrophytes.

In some embodiments, the fungus is in the genus Microsporum (e.g., Microsporum gypseum, M. amazonicum, M. audouinii, M. boullardii, M. canis, M. canis, M. cookei, M. distortum, M. duboisii, M. equinum, M. ferrugineum, M. fulvum, M. gallinae, M. langeronii, M. nanum, M. persicolor, M. praecox, M. ripariae, or M. rivalieri).

In some embodiments, the fungus is in the genus Epidermophyton (e.g., Epidermophyton floccosum or E. stockdaleae).

In some embodiments, the onychomycosis is a non-dermatophytic onychomycosis. In some embodiments, the non-dermatophytic onychomycosis is caused by Candida albicans, C. glabrata, C. dubliniensis, C. krusei, C. parapsilosis, C. tropicalis, C. orthopsilosis, C. guilliermondii, C. rugosa, or C. lusitaniae. In some embodiments, the non-dermatophytic onychomycosis is caused by Candida albicans.

In some embodiments of the methods described herein, the onychomycosis is in the nail plate, nail matrix, nail bed, nail cuticle, nail lunula, nail root, nail sinus, nail hyponychium, nail free margin, or any combination thereof.

In some embodiments of the methods described herein, the onychomycosis is distal subungual onychomycosis, white superficial onychomycosis, proximal subungual onychomycosis, candidal onychomycosis, or total dystrophic onychomycosis.

In some embodiments of the methods described herein, the subject has failed treatment with an oral therapy for onychomycosis. In some embodiments, the subject has failed treatment with terbinafine, itraconazole, fluconazole, posaconazole, and/or griseofulvin.

In some embodiments of the methods described herein, the subject has failed treatment with a topical therapy for onychomycosis. In some embodiments, the subject has failed treatment with ketoconazole, miconazole, butenafine, and/or naftifine.

In some embodiments of the methods described herein, the subject has failed treatment with a nonpharmacological therapy for onychomycosis. In some embodiments, the subject as failed mechanical, chemical, or surgical nail avulsion, laser treatment, and/or photodynamic treatment.

In some embodiments of the methods described herein, the method further includes administering to the subject at least one antifungal agent (e.g., one, two, or three antifungal agents) in combination with CD101 in salt or neutral form.

In some embodiments, CD101 in salt or neutral form and the antifungal agent are administered substantially simultaneously.

In some embodiments, CD101 in salt or neutral form and the antifungal agent are administered separately. In some embodiments, CD101 in salt or neutral form is administered first, followed by administration of the antifungal agent. In some embodiments, the antifungal agent is administered first, followed by administration of CD101 in salt or neutral form.

In some embodiments, CD101 in salt or neutral form and the antifungal agent are administered substantially simultaneously, followed by administration of CD101 in salt or neutral form or the antifungal agent alone.

In some embodiments, CD101 in salt or neutral form or the antifungal agent is administered alone, followed by administering of CD101 in salt or neutral form and the antifungal agent substantially simultaneously.

In some embodiments, the antifungal agent is an allylamine compound, an azole compound, an echinocandin compound, a polygene compound, flucytosine (Ancobon®)), enfumafungin, or SCY-078. In some embodiments, the antifungal agent is terbinafine, itraconazole, fluconazole, posaconazole, griseofulvin, ketoconazole, miconazole, butenafine, or naftifine.

In some embodiments of the methods described herein, the subcutaneous administration of CD101 in salt or neutral form does not cause any negative injection site effects (e.g., pain or tenderness at the injection site, itching, bruising, and/or swelling of the skin, skin rash, allergic reactions at the injection site).

In some embodiments of the methods described herein, CD101 is administered as an aqueous pharmaceutical composition (e.g., an aqueous pharmaceutical composition having a pH of from 4 to 8).

In some embodiments of the methods described herein, CD101 in salt form is CD101 acetate. In any of the above methods, CD101 can be substituted with a CD101 analog, such as compound 2 (described herein) in salt or neutral form or a compound described in U.S. Pat. No. 9,217,014 or U.S. Application No. 62/174,815, incorporated herein by reference. For example, CD101 could be substituted with a compound described in U.S. Pat. No. 9,217,014, such as compounds described by formulas (I), (Ia), (Ib), (II), (IIa), (IIb), or (III), wherein the compound is not CD101; including compound 5, compound 6, compound 7, compound 12, compound 15, compound 16, compound 17, compound 18, compound 23, compound 24, or pharmaceutically acceptable salts thereof. As a further example, CD101 can be substituted with compounds described in U.S. Application No. 62/174,815, such as compounds described by formulas (Ia), (Ib) or (Ic), wherein the compound is not CD101.

### Definitions

As used herein, the term “CD101” refers to the compound having the structure shown below. The term “CD101 in salt form” or “a salt of CD101” refers to CD101 when its tertiary ammonium ion positive charge is balanced with a negative counterion (e.g., an acetate).

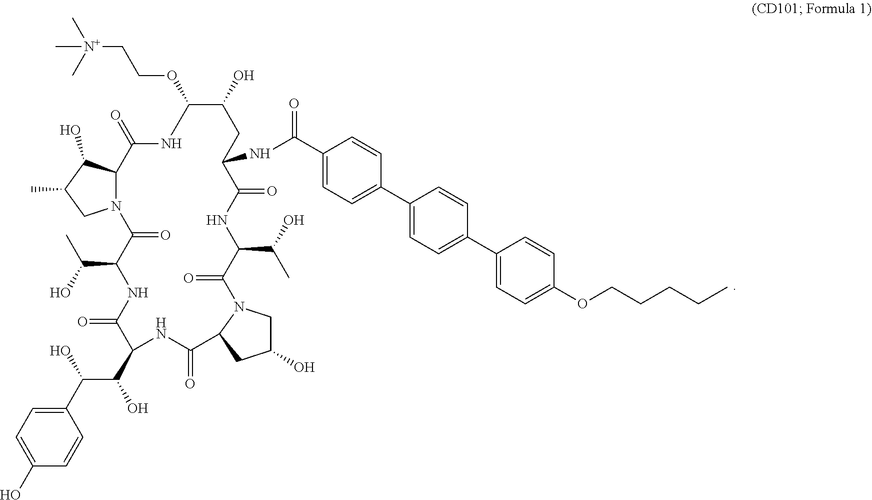 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1]

As used herein, the term “compound 2” refers to a salt of the compound of Formula 2, or a neutral form thereof. Compound 2 has a structure (below) in which the tertiary ammonium ion positive charge of the compound in Formula 2 is balanced with a negative counterion (e.g., an acetate) in its salt form. The structure of compound 2 is depicted below.

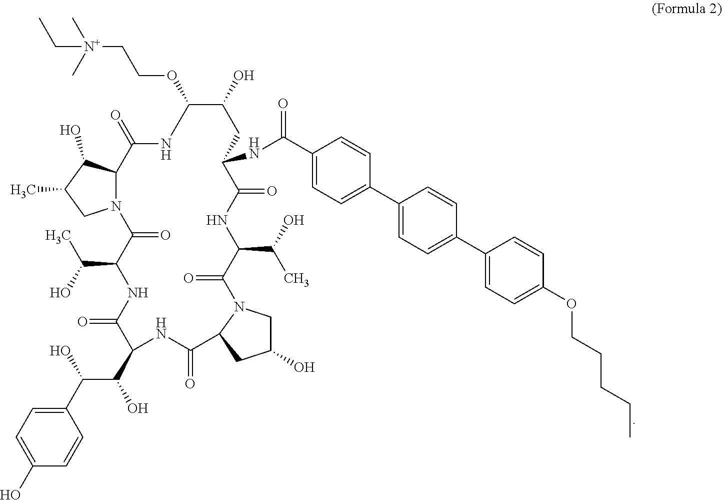 [SMILES: CCCCCOc1ccc(-c2ccc(-c3ccc(C(=O)N[C@H]4C[C@@H](O)[C@@H](OCC[N+](C)(C)CC)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1]

As used herein, the term “CD101 analog” refers to compound 2 (described herein) in salt or neutral form or a compound described in U.S. Pat. No. 9,217,014 or U.S. Application No. 62/174,815, each incorporated herein by reference. For example, CD101 analogs include compounds described in U.S. Pat. No. 9,217,014, such as compounds described by formulas (I), (Ia), (Ib), (II), (IIa), (IIb), or (III); including compound 5, compound 6, compound 7, compound 12, compound 15, compound 16, compound 17, compound 18, compound 23, compound 24, and pharmaceutically acceptable salts thereof. As a further example, CD101 analogs also include compounds described in U.S. Application No. 62/174,815, such as compounds described by formulas (Ia), (Ib) or (Ic).

As used herein, the term “a neutral form” of CD101 or a CD101 analog (e.g., compound 2) includes the zwitterionic forms of CD101 (the compound of Formula 1) or the CD101 analog (e.g., compound 2), respectively, in which the compound has no net positive or negative charge. The zwitterion is present in a higher proportion in basic medium (e.g., pH 9) relative to CD101, the CD101 analog (e.g., compound 2), or a salt thereof. In some embodiments, the zwitterion may also be present in its salt form.

As used herein, the term “dermatophytosis” or “dermatophyte infection” refers to an infection caused by dermatophytes, which are fungi that require keratin for growth. Dermatophytes are fungi in the genus Microsporum, Epidermophyton, and Trichophyton. These fungi can cause superficial infections of the skin, hair, and/or nails. Onychomycosis (further defined herein), when caused by fungi in the genus Microsporum, Epidermophyton, or Trichophyton, is a form of dermatophytosis (also referred to as “dermatophytic onychomycosis” herein). Dermatophytes are spread by direct contact from other people (anthropophilic organisms), animals (zoophilic organisms), and soil (geophilic organisms), as well as indirectly from fomites.

As used herein, the term “onychomycosis” refers a fungal infection of the nail (e.g., toe nails or finger nails). Some forms of onychomycosis are dermatophyte infections when the onychomycosis is caused by fungi in the genus Microsporum, Epidermophyton, or Trichophyton (also referred to as “dermatophytic onychomycosis” herein). For example, tinea unguium specifically describes a dermatophytic onychomycosis of the nail plate. Other forms of onychomycosis may be caused by fungi in other genera (e.g., Candida) (also referred to as “non-dermatophytic onychomycosis”).

As used herein, the term “antifungal agent” refers to an antifungal drug used to treat a fungal infection. Antifungal drugs include, but are not limited to, allylamine compounds, azole compounds, echinocandin compounds, polyene compounds, flucytosine (Ancobon®)), enfumafungin, SCY-078, and APX001. In some embodiments of the methods described herein, the subject may be administered both CD101 or a CD101 analog in salt or neutral form and at least one antifungal agent for the duration of the treatment. An antifungal agent as defined herein does not include CD101 or a CD101 analog in salt or neutral form.

As used herein, the term “oral therapy” refers to drugs (e.g., antifungal agents) that are formulated for oral administration (e.g., oral suspension, oral tablet, oral capsule, oral solution, oral granules). Examples of antifungal agents that are formulated for oral administration include, e.g., terbinafine, itraconazole, fluconazole, posaconazole, and griseofulvin.

As used herein, the term “topical therapy” refers to drugs (e.g., antifungal agents) that are formulated for topical administration (e.g., topical solution, topical cream, topical lotion, topical gel, topical foam). Examples of antifungal agents that are formulated for topical administration include, e.g., ketoconazole, miconazole, butenafine, and naftifine.

As used herein, the term “nonpharmacological therapy” refers to treatments that do not involve the administration of drugs or treatments that involve the administration of drugs in combination with other non-drug related therapy. Examples of nonpharmacological therapies for onychomycosis include, e.g., mechanical, chemical, or surgical nail avulsion, laser treatment, and photodynamic treatment.

As used herein, the term “photodynamic treatment” refers to treatments that use light to excite small molecule compounds, which is then activated to produce therapeutic effects. For example, protoporphyrin IX is an effective inhibitor of Trichophyton rubrum when used in conjunction with red-spectrum light. In some embodiments, the small molecule compound may be activated by light to produce reactive oxygen species (ROS), thus, leading to selective tissue destruction.

As used herein, to “prevent” or “reduce the likelihood” of dermatophytosis (e.g., dermatophytic onychomycosis) or non-dermatophytic onychomycosis (e.g., onychomycosis caused by fungi in the genus Candida) refers to prophylactic treatment of a subject who is not yet ill, but who is susceptible to, or otherwise at risk of, developing the dermatophytosis or non-dermatophytic onychomycosis.

As used herein, the term “negative injection site effect” refers to any adverse reactions the subject might experience at the site of the subcutaneous injection after a drug is administered. Negative injection site effects at the injection site may include, e.g., pain or tenderness at the injection site, itching, bruising, and/or swelling of the skin, and skin rash. Some negative injection site effects may be caused by an allergic reaction to the drug or other components in the pharmaceutical composition.

As used herein, the term “about” refers to a range of values that is ±10% of specific value. For example, “about 150 mg” includes ±10% of 150 mg, or from 135 mg to 165 mg. Such a range performs the desired function or achieves the desired result. For example, “about” may refer to an amount that is within less than 10% of, within less than 5% of, within less than 1% of, within less than 0.1% of, and within less than 0.01% of the stated amount.

By “dose” is meant the amount of CD101 or a CD101 analog administered to the subject (e.g., a human). As used herein, the amount in each dose refers to the amount of CD101 (structure shown above) or a CD101 analog that does not include the negative counterion (e.g., an acetate) if CD101 or the CD101 analog is in its salt form. For example, a dose of about 25 mg to about 600 mg of CD101 or a CD101 analog in salt or neutral form refers to about 25 mg to about 600 mg of CD101 or the CD101 analog, not including the acetate ion if CD101 or the CD101 analog is in an acetate salt form.

All publications, patents, and patent applications are herein incorporated by reference in their entirety to the same extent as if each individual publication, patent or patent application was specifically and individually indicated to be incorporated by reference in its entirety.

### DESCRIPTION OF THE DRAWINGS

FIG. 1 shows the clinical efficacy of CD101 (administered at 35 mg/kg daily or once a week) and terbinafine in the treatment of Trichophyton mentagrophytes dermatophytosis in a guinea pig model.

FIG. 2 shows the mycological efficacy of CD101 (administered at 35 mg/kg daily or once a week) and terbinafine in the treatment of Trichophyton mentagrophytes dermatophytosis in a guinea pig model.

FIGS. 3A-3E show the clinical appearances of guinea pig skin after administering CD101 at 35 mg/kg daily or once a week or terbinafine.

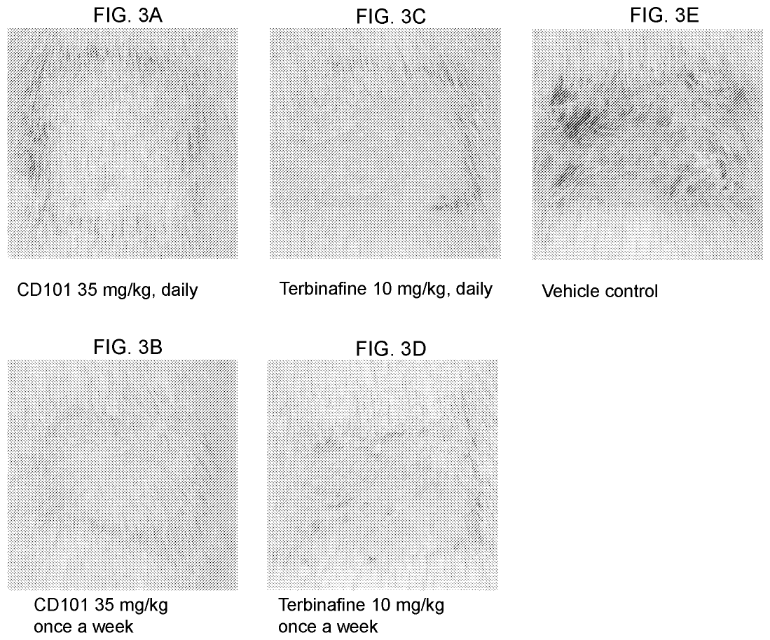

FIG. 4 shows the clinical efficacy of CD101 (administered at 10, 20, or 40 mg/kg once a week) and terbinafine in the treatment of Trichophyton mentagrophytes dermatophytosis in a guinea pig model.

FIG. 5 shows the mycological efficacy of CD101 (administered at 10, 20, or 40 mg/kg once a week) and terbinafine in the treatment of Trichophyton mentagrophytes dermatophytosis in a guinea pig model.

FIGS. 6A-6E show the clinical appearances of guinea pig skin after administering CD101 at 10, 20, or 40 mg/kg once a week or terbinafine.

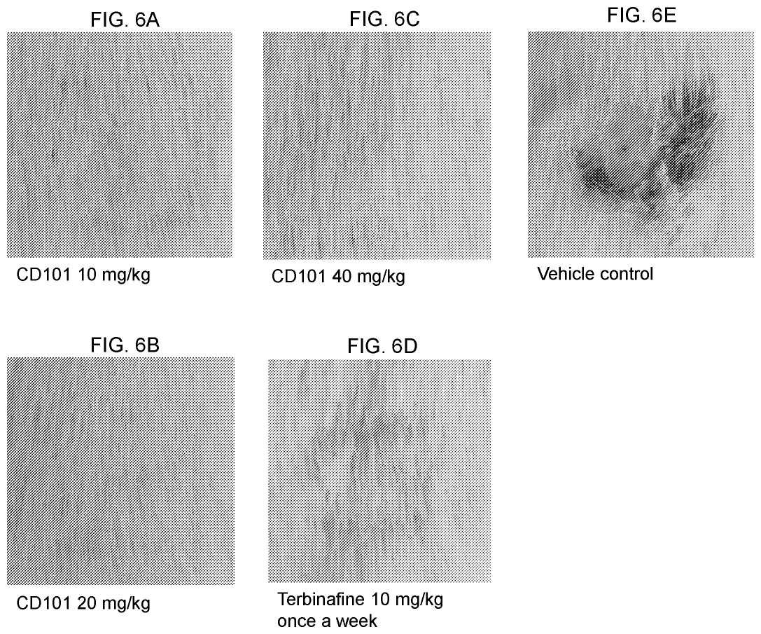

FIG. 7 shows the individual and mean concentrations of CD101 in the fingernails of monkeys subcutaneously injected with CD101.

FIG. 8 shows the individual and mean concentrations of CD101 in the fingernail beds of monkeys subcutaneously injected with CD101.

FIG. 9 shows the individual and mean concentrations of CD101 in the toenail of monkeys subcutaneously injected with CD101.

FIG. 10 shows the individual and mean concentrations of CD101 in the toenail beds of monkeys subcutaneously injected with CD101.

FIG. 11 shows the individual and mean concentrations of CD101 in the skin of monkeys subcutaneously injected with CD101.

### DETAILED DESCRIPTION

Provided are methods of treating or preventing dermatophytosis (e.g., dermatophytic onychomycosis) in a subject (e.g., a human) by administering (e.g., subcutaneously administering) to the subject a salt of CD101, a CD101 analog, or a neutral form thereof. In some embodiments, the methods also relate to treating or preventing non-dermatophytic onychomycosis (e.g., onychomycosis caused by fungi in the genus Candida). In some embodiments of the methods described herein, CD101 or a CD101 analog in salt or neutral form is administered in combination with at least one antifungal agent. The inventors have found that CD101 displays long-acting pharmacokinetics with a long half-life, slow clearance, and strong and fast-acting activities against dermatophytes (e.g., Trichophyton, Microsporum, Epidermophyton) and fungi in other genera (e.g., Candida). The stability of CD101 in salt or neutral form especially enables non-intravenous formulations of CD101, e.g., subcutaneous formulations.

### I. Cutaneous Fungal Infections

Cutaneous fungal infections are infections of the skin, hair, and/or nails caused by a fungus. Cutaneous fungal infections may present as either a superficial or deep infection of the skin, hair, and/or nails. Dermatophytosis is a type of cutaneous fungal infection caused by dermatophytes, which are fungi in the genera Trichophyton, Microsporum, and Epidermophyton. The duration of cutaneous fungal infections can be acute, subacute, or chronic. Acute cutaneous fungal infections are generally severe and sudden in onset and usually lasts a short term (e.g., up to one week). Subacute cutaneous fungal infections often last a few weeks (e.g., more than one week; one to 12 weeks). Chronic cutaneous fungal infections usually last three months or more. In any of acute, subacute, and chronic cutaneous fungal infections, the cutaneous fungal infection may disappear and then reoccur. For example, in acute cutaneous fungal infections, symptoms may disappear for a few hours or a day, then appear again.

The severity of symptoms of cutaneous fungal infections may vary greatly depending on factors such as the type of cutaneous fungal infection and its duration. The severity is often characterized as mild when the symptoms are itching, skin thickening, skin cracking, and dry and scaly skin. Skin changes in mild cutaneous fungal infections are predominately visual and have minimal impact on patients' ability to perform daily activities. The severity is often characterized as moderate when the symptoms include burning, pain, inflammation, and plague formation in the skin and abscess in the skin. The structural changes in the nail or skin often take a long time to restore. Patients with moderate cutaneous fungal infections are still able to achieve remission within weeks and perform daily activities. The severity is often characterized as severe when the symptoms include nodular or pustular lesions, disseminated infection, and discharges or any other non-life threatening symptoms. Patients with severe cutaneous fungal infections may take months or years to achieve remission and may have difficulties performing daily activities. Treatments for severe cutaneous fungal infections may include surgery. The severity is often characterized as very severe when the infection could result in tumefaction, surgery, septicemia, and possibly death. Table 1 below provides a summary of some examples of indications that are acute, subacute, and chronic cutaneous fungal infections and their causative mycoses.

TABLE 1

| Indication | Duration | Causative mycoses | Severity |
|---|---|---|---|
| Cutaneous blastomycosis | acute | Blastomyces dermatitidis | Severe |
| Cutaneous aspergillosis | acute | Aspergillus fumigatus, Aspergillus flavus | Very severe |
| Tinea oedis | acute or chronic | Trichophyton rubrum, Trichophyton | Moderate |
| | | interdigitale, Epidermophyton floccosum | |
| Candidiasis of vulva and | acute or chronic | Candida albicans | Severe |
| vagina | | | |
| Tinea corporis | acute or chronic | Trichophyton mentagrophytes, | Moderate |
| | | Trichophyton rubrum, Microsporum canis | |
| Dermatophytid reaction | acute or chronic | Any dermatophyte | Severe |
| Tinea cruris | acute or chronic | Trichophyton rubrum, Trichophyton | Moderate |
| | | mentagrophytes | |
| Tinea capitis | acute or chronic | Trichophyton tonsurans, Microsporum | Moderate |
| | | canis, Microsporum audouinii, | |
| | | Trichophyton schoenleinii, Trichophyton | |
| | | violaceum | |
| Tinea manuum | acute or chronic | Trichophyton erinacei, Trichophyton | Moderate |
| | | verrucosum, Microsporum canis, | |
| | | Microsporum gypseum | |
| Cutaneous and | acute or chronic | Rhizopus oryzae, Conidiobolus coronatus, | Very Severe |
| subcutaneous zygomycosis | | Conidiobolus incongruous, Basidiobolus | |
| | | ranarum | |
| Candidal balanitis | acute or chronic | Candida albicans | Moderate |
| Candidal intertrigo | acute or chronic | Candida albicans | Moderate |
| Cutaneous | acute or chronic | Coccidioides immitis | Mild |
| coccidioidomycosis | | | |
| Tinea barbae | acute or chronic | Trichophyton rubrum | Moderate |
| Otomycosis | Subacute or | Aspergillus niger, Candida spp. | Mild |
| | chronic | | |
| Cutaneous sporotrichosis | Subacute or | Sporothrix schenckii | Severe |
| | chronic | | |
| Tinea unguium | chronic | Trichophyton rubrum, Trichophyton | Severe |
| | | interdigitale | |
| Tinea versicolor | chronic | Malassezia furfur | Mild |
| Candidiasis of nails | chronic | Candida albicans | Severe |
| Mycetoma | chronic | Madurella, Acremonium, | Very Severe |
| | | Pseudallescheria, Exophiala, | |
| | | Leptosphaeria, Curvularia, Fusarium, | |
| | | Aspergillus species | |
| Chromomycosis | chronic | Fonsecaea pedrosoi, Phialophora | Severe |
| | | verrucosa, Cladosporium carrionii, | |
| | | Fonsecaea compacta | |
| Chronic mucocutaneous | chronic | Candida albicans | Severe |
| candidiasis | | | |
| Cutaneous cryptococcosis | chronic | Cryptococcus neoformans | Very Severe |
| Tinea imbricata | chronic | Trichophyton concentricum | Moderate |
| Tinea nigra | chronic | Hortaea werneckii | Mild |
| Malassezia folliculitis | chronic | Malassezia furfur, Malassezia | Moderate |
| | | pachydermatous, Malassezia sympodialis, | |
| | | Malassezia globosa, Malassezia obtusa, | |
| | | Malassezia restricta, Malassezia slooffiae | |
| Piedra | chronic | Piedraia hortae, Trichosporon asahii, | Moderate |
| | | Trichosporon ovoides | |
| Cutaneous cryptococcosis | chronic | Cryptococcus neoformans | Very Severe |

### II. Dermatophytosis

Dermatophytosis, also referred to as a ringworm or tinea infection, is a fungal infection of the skin caused by dermatophytes, which are fungi in the genus Microsporum, Epidermophyton, and Trichophyton. Fungi in the genus Trichophyton include, e.g., Trichophyton rubrum, T. mentagrophytes, T. interdigitale, T. violaceum, T. tonsurans, T. soudanense, T. concentricum, T. megnini, T. schoenleinii, T. yaoundei, T. verrucosum, T. simii, T. redellii, T. equinum, T. ajelloi, T. vanbreuseghemii, T. terrestre, T. phaseoliforme, T. flavescens, T. gloriae, and T. onychocola. Fungi in the genus Microsporum include, e.g., Microsporum gypseum, M. amazonicum, M. audouinii, M. boullardii, M. canis, M. canis, M. cookei, M. distortum, M. duboisii, M. equinum, M. ferrugineum, M. fulvum, M. gallinae, M. langeronii, M. nanum, M. persicolor, M. praecox, M. ripariae, and M. rivalieri. Fungi in the genus Epidermophyton include, e.g., Epidermophyton floccosum and E. stockdaleae. Dermatophytes are spread by direct contact from other people (anthropophilic organisms), animals (zoophilic organisms), and soil (geophilic organisms), as well as indirectly from fomites. Dermatophytes require keratin for growth and either invade or grow in keratin. These fungi can cause superficial or deep infections of the skin, hair, and/or nails.

Parts of the body that are likely to be affected by dermatophytosis include, e.g., scalp, feet, hands, nail, beard area, and groin. Some types of dermatophytosis are clinically classified according to the infected body part, e.g., tinea pedis (foot), tinea unguium (nails) (a form of dermatophytic onychomycosis), tinea manuum (hand), tinea cruris (groin), tinea corporis or tinea circinata (body), tinea capitis (scalp), tinea faciei (face), and tinea barbae (beard). Other types of dermatophytosis include, e.g., tinea imbricate. Symptoms and signs of dermatophytosis vary depending on the specific type of infection and may include, e.g., red, scaly, itchy, and/or raised patches on the skin, patches on the skin that ooze or develop blisters, bald patches on the scalp, and thickening and discoloration of nails. Tinea manuum often occurs together with tinea pedis. Signs of tinea manuum include scaling and redness of the hand. Tinea unguium (a form of dermatophytic onychomycosis) appears as thickening, discoloration, and cracking of the nail and separation of the nail from the nail bed. Tinea cruis is more common in men and appears as erythematous with a central clearing and a raised edge. Signs of tinea barbae include redness, scaling, and pustules in the beard area. Tinea faciei generally appears as a red rash on the face, followed by patches of small, raised bumps. Other tinea infections are described in detail below.

Tinea Pedis

Tinea pedis (also referred to as Foot Ringworm or Athlete's Foot) is the most common dermatophytosis because moisture resulting from foot sweating facilitates fungal growth. Tinea pedis is most frequently caused by Trichophyton rubrum, Trichophyton interdigitale, or Epidermophyton floccosum and may occur as any of four clinical forms or in combination: chronic hyperkeratotic, chronic intertriginous, acute ulcerative, and vesiculobullous. Tinea pedis commonly affects the plantar surface of the foot and the web of the toe where the skin may be macerated and erythematous. The most common symptom is cracked, flaking, and peeling skin between the toes or on the side of the foot. Other symptoms can include red and itchy skin, burning or stinging pain, and blisters that ooze or get crusty. If the fungus spreads to the nails, the nails can become discolored, thick, and even crumble. Tinea pedis may occur at the same time as other fungal or yeast skin infections. In some embodiments, tinea pedis may respond to drugs or self-care, although reoccurrence is common. Long-term medicine and preventive measures may be needed.

The high prevalence rate of tinea pedis has been linked to increased urbanization and use of community showers, sports, and the use of occlusive footwear. In some embodiments, immunocompromized conditions may drive epidemiology. These factors are thought to contribute to the high prevalence of tinea pedis in certain occupational groups: marathon runners (22%-31% prevalence), miners (21%-72.9% prevalence), soldiers (16.4%-58% prevalence). Developed countries have high rates of tinea pedis. Tinea pedis affects approximately 26.5 million people in the U.S. per year. Pharmaceutical products for the treatment of tinea pedis include terbinafine (oral and topical), butenafine (topical), miconazole (topical), and itraconazole (oral). In some embodiments, systemic treatment may be used if there is co-existing infection of the nail (tinea unguium).

Tinea Corporis
Tinea corporis is a dermatophytosis that causes pink-to-red annular (O-shaped) patches and plaques with raised scaly borders that expand peripherally and tend to clear centrally. A rare variant form appears as nummular (circle- or round-shaped) scaling patches studded with small papules or pustules that have no central clearing. Main causes for tinea corporis are Trichophyton mentagrophytes, Trichophyton rubrum, and Microsporum canis. Tinea corporis often begins as a pruritic, circular or oval, erythematous, scaling patch, or plaque that spreads centrifugally. Central clearing follows, while an active, advancing, raised border remains. The result is an annular (ring-shaped) plaque from which the disease derives its common name (ringworm). Multiple plaques may coalesce. Chronic tinea corporis tends to be most prominent in body folds. Typically skin lesions are on exposed skin of the trunk, arms, and legs. Tinea corporis contracted from infected animals, particularly kittens and puppies, is often intensely inflammatory. Extensive tinea corporis should raise concern for an underlying immune disorder, such as HIV, or for diabetes.

Tinea corporis may be treated orally or topically. Oral therapy is often recommended if the infection is extensive, severe, or reoccurring. Pharmaceutical products for the treatment of tinea corporis include terbinafine and itraconazole.

Tinea Capitis

Tinea capitis is a dermatophytosis that causes the gradual appearance of round patches of dry scale, alopecia, or both. Main causes for tinea capitis are Trichophyton tonsurans, Microsporum canis, Microsporum audouinii, Trichophyton schoenleinii, and Trichophyton violaceum. Trichophyton tonsurans infection causes black dot ringworm, in which hair shafts break at the scalp surface. Microsporum audouinii infection causes gray patch ringworm, in which hair shafts break above the surface, leaving short stubs. Trichophyton schoenleinii causes a chronic form of tinea capitis that is usually acquired before adolescence and extending into adulthood. In some embodiments, tinea capitis also manifests as diffuse scaling or a diffuse pustular pattern. The other symptoms of tinea capitis include kerion (very inflamed mass) and favus (yellow crusts and matted hair).

Tinea capitis is often treated with systemic antifungal agents because topical antifungal agents do not penetrate the hair shaft. In some embodiments, concomitant treatment with both oral and topical agents is used. Oral treatments for tinea capitis include, e.g., griseofulvin, terbinafine, fluconazole, and itraconazole. Topical treatments for tinea capitis include, e.g., selenium sulfide and ketoconazole. In some embodiments, relapse rates for tinea capitis are high because of rapid clearance of the drug from the skin with the cessation of treatment.

### III. Onychomycosis

Onychomycosis is a fungal infection of the nail (e.g., toenails or fingernails). Onychomycosis may be dermatophytic onychomycosis or non-dermatophytic onychomycosis. Dermatophytic onychomycosis is a form of onychomycosis that is caused by dermatophytes, e.g., fungi in the genus Microsporum, Epidermophyton, or Trichophyton. For example, tinea unguium specifically describes a dermatophytic onychomycosis of the nail plate. Non-dermatophytic onychomycosis refers to forms of onychomycosis caused by fungi in other genera, e.g., Candida.

Onychomycosis may affect any component of the nail, e.g., nail plate, nail matrix, nail bed, nail cuticle, nail lunula, nail root, nail sinus, nail hyponychium, nail free margin, or any combination thereof. There are five main subtypes of onychomycosis: distal subungual onychomycosis, white superficial onychomycosis, proximal subungual onychomycosis, candidal onychomycosis, and total dystrophic onychomycosis. In some embodiments, a subject may suffer from one or a combination of these subtypes of onychomycosis.

Signs of onychomycosis vary among subjects and are different among the subtypes. Distal subungual onychomycosis is the most common form of onychomycosis and is often caused by Trichophyton rubrum, which invades the nail bed and the underside of the nail plate. The infecting fungi migrate proximally through the underlying nail matrix. Distal subungual onychomycosis appears as focal parakeratosis and subungual hyperkeratosis. In some embodiments, distal subungual onychomycosis results in onycholysis (detachment of the nail plate from the nail bed). White superficial onychomycosis is caused by fungal invasion of the superficial layers of the nail plate to form “white islands” on the plate. The most common causative fungus in white superficial onychomycosis is Trichophyton mentagrophytes. White superficial onychomycosis often confined to the toenails and appears as small, white, speckled, or powdery patches on the surface of the nail plate. The nail often becomes roughened and crumbles easily in white superficial onychomycosis. Proximal subungual onychomycosis is often caused by fungal infection of the newly formed nail plate through the proximal nail fold on the lunula area and migrates distally with nail growth. Symptoms of proximal subungual onychomycosis include, e.g., subungual hyperkeratosis, proximal onycholysis, leukonychia, and destruction of the proximal nail plate. Trichophyton rubrum is the principal causative agent of proximal subungual onychomycosis. Candidal onychomycosis is Candida species invasion of the nails and often develops in patients with chronic mucocutaneous candidiasis or immunodepression. Candida albicans is the main causative agent. Candidal onychomycosis often affects the entire nail plate and the soft tissue around the nail and causes onycholysis and paronychia. Total dystrophic onychomycosis is the most advanced form of any subtype of onychomycosis and presents as a thickened, opaque, and yellow-brown nail.

### IV. Combination Therapy

In some embodiments, in addition to administering CD101 or a CD101 analog in salt or neutral form to treat or prevent a subject from dermatophytosis (e.g., dermatophytic onychomycosis) or non-dermatophytic onychomycosis, the subject may also be administered at least one antifungal agent. The methods described herein further include administering at least one antifungal agent (e.g., one, two, or three antifungal agents) in combination with CD101 or a CD101 analog in salt or neutral form. In some embodiments of the methods, CD101 or a CD101 analog in salt or neutral form and the antifungal agent are administered substantially simultaneously. In some embodiments, CD101 or a CD101 analog in salt or neutral form and the antifungal agent are administered separately. For example, CD101 or a CD101 analog in salt or neutral form may be administered first, followed by administration of the antifungal agent. In other embodiments, the antifungal agent is administered first, followed by administration of CD101 or a CD101 analog in salt or neutral form. In some embodiments, CD101 or a CD101 analog in salt or neutral form and the antifungal agent are administered substantially simultaneously, followed by administration of CD101 in salt or neutral form or the antifungal agent alone. In other embodiments, CD101 or a CD101 analog in salt or neutral form or the antifungal agent is administered alone, followed by administration of CD101 or a CD101 analog in salt or neutral form and the antifungal agent substantially simultaneously. Additionally or alternatively, in any of the monotherapies or combination therapies described herein, the subject may also receive appropriate source control interventions, including debridement of damaged tissue, nail trimming, laser therapies, photodynamic therapies, and/or surgical correction of the underlying pathology.

Depending on the administration methods, in some embodiments, CD101 or a CD101 analog in salt or neutral form may be formulated in a pharmaceutical composition alone. In some embodiments, CD101 or a CD101 analog in salt or neutral form may be formulated in combination with at least one antifungal agent in the same pharmaceutical composition.

Antifungal agents that may be administered in combination with CD101 or a CD101 analog in salt or neutral form in methods described herein include, but are not limited to, allylamine compounds, azole compounds, echinocandin compounds, polyene compounds, flucytosine (Ancobon®)), enfumafungin, SCY-078, and APX001. Allylamine compounds include, but are not limited to, terbinafine, butenafine, naftifine, and amorolfine. Azole compounds include, but are not limited to, fluconazole, albaconazole, bifonazole, butoconazole, clotrimazole, econazole, efinaconazole, fenticonazole, isavuconazole, isoconazole, itraconazole, ketoconazole, luliconazole, miconazole, omoconazole, oxiconazole, posaconazole, pramiconazole, ravuconazole, sertaconazole, sulconazole, terconazole, tioconazole, voriconazole, VT-1161, and VT-1598. Echinocandin compounds include, but are not limited to, micafungin, caspofungin, anidulafungin, cilofungin, echinocandin B, and pneumocandin (but not CD101 in salt or neutral form). Polyene compounds include, but are not limited to, amphotericin B, nystatin, natamycin, rimocidin, filipin, candicin, hamycin, perimycin, and dermostatin.

Furthermore, in some embodiments of the methods described herein, CD101 or a CD101 analog in salt or neutral form may be administered in combination with one or more of the antifungal agents for the treatment or prevention of the indication as listed in Table 2.

TABLE 2

| | | Route of | |
|---|---|---|---|
| Antifungal agent(s) | Brand name | administration | Indication(s) |
| ketoconazole | KETOZOLE | Topical cream | Tinea pedis, Tinea cruris, Tinea corporis |
| luliconazole | LUZU | Topical cream | Tinea pedis, Tinea cruris, Tinea corporis |
| luliconazole | LUZU | Topical solution | Tinea unguium |
| miconazole | MONISTAT- | Topical cream | Tinea pedis, Tinea cruris, Tinea corporis |
| | DERM | | |
| oxiconazole | OXISTAT | Topical cream | Tinea pedis, Tinea cruris, Tinea corporis |
| oxiconazole | OXISTAT | Topical lotion | Tinea pedis, Tinea cruris, Tinea corporis |
| posaconazole | NOXAFIL/SCH | Oral suspension | Tinea unguium |
| | 56592 | | |
| efinaconazole | JUBLIA | Topical solution | Tinea unguium |
| itraconazole | ONMEL | Oral tablet | Tinea unguium |
| itraconazole | SPORANOX | Oral capsule | Tinea unguium |
| itraconazole | SPORANOX | Oral solution | Tinea unguium |
| clotrimazole & | LOTRISONE | Topical cream | Tinea pedis, Tinea cruris, Tinea corporis |
| betamethasone | | | |
| clotrimazole & | LOTRISONE | Topical lotion | Tinea pedis, Tinea cruris, Tinea corporis |
| betamethasone | | | |
| ciclopirox olamine | PENLAC | Topical solution | Tinea unguium |
| ciclopirox olamine | LOPROX | Topical cream | Tinea pedis, Tinea cruris, Tinea corporis |
| ciclopirox olamine | LOPROX | Topical | Tinea pedis, Tinea corporis, Tinea cruris |
| | | suspension | |
| ciclopirox olamine | LOPROX | Topical gel | Tinea pedis, Tinea cruris, Tinea corporis |
| griseofulvin microsize | GRIFULVIN V | Oral tablet | Tinea pedis, Tinea cruris, Tinea corporis, |
| | | | Tinea capitis, Tinea barbae |
| griseofulvin | GRIS-PEG | Oral tablet | Tinea unguium, Tinea pedis, Tinea cruris, |
| ultramicrosize | | | Tinea corporis, Tinea capitis, Tinea |
| | | | barbae |
| griseofulvin | griseofulvin | Oral suspension | Tinea unguium |
| ciclopirox olamine | PENLAC | Topical solution | Tinea unguium |
| ciclopirox olamine | LOPROX | Topical cream | Tinea pedis, Tinea cruris, Tinea corporis |
| terbinafine | LAMISIL | Oral tablet | Tinea unguium |
| terbinafine | LAMISIL | Oral granules | Tinea capitis |
| butenafine | MENTAX | Topical cream | Tinea pedis, Tinea cruris, Tinea corporis |
| naftifine | NAFTIN | Topical gel | Tinea pedis |
| naftifine | NAFTIN | Topical cream | Tinea pedis, Tinea cruris, Tinea corporis |
| tavaborole | KERYDIN | Topical solution | Tinea unguium |
| econazole nitrate | ECOZA | Topical foam | Tinea pedis |
| econazole nitrate | SPECTAZOLE | Topical cream | Tinea pedis, Tinea cruris, Tinea corporis |
| sertaconazole nitrate | ERTACZO | Topical cream | Tinea pedis |
| sulconazole nitrate | EXELDERM | Topical cream | Tinea cruris, Tinea corporis |
| sulconazole nitrate | EXELDERM | Topical solution | Tinea cruris, Tinea corporis |
| ME1111 | | Topical solution | Tinea unguium |
| NVXT solution | NOVEXATIN | Topical solution | Tinea unguium |
| VT-1161 | | Oral tablet | Tinea unguium |
| VT-1161 | | Oral tablet | Tinea pedis |

### V. Methods of Treatment

The invention provides methods of treating dermatophytosis in a subject by administering to the subject CD101 or a CD101 analog in salt or neutral form thereof. The invention also provides methods of treating onychomycosis (e.g., dermatophytic onychomycosis and non-dermatophytic onychomycosis) in a subject by administering (e.g., subcutaneously administering) to the subject CD101 or a CD101 analog in salt or neutral form thereof.

In some embodiments of the methods described herein, the subject has failed an oral therapy for the dermatophytosis or onychomycosis. In some embodiments, the subject has failed treatment with terbinafine, itraconazole, fluconazole, posaconazole, and/or griseofulvin. In some embodiments of the methods described herein, the subject has failed a topical therapy for the dermatophytosis or onychomycosis. In some embodiments, the subject has failed treatment with ketoconazole, miconazole, butenafine, and/or naftifine. In some embodiments, the subject has failed treatment with one or more of the antifungal agents listed in Table 2 for the dermatophytosis or onychomycosis. In some embodiments of the methods to treat onychomycosis, the subject has failed treatment with a nonpharmacological therapy, such as laser treatment, photodynamic treatment, and/or mechanical, chemical, or surgical nail avulsion.

Furthermore, the invention also provides methods of preventing or reducing the likelihood of dermatophytosis (e.g., dermatophytic onychomycosis) or non-dermatophytic onychomycosis in a subject by subcutaneously administering to the subject CD101 or a CD101 analog in salt or neutral form. In some embodiments, the subject is in a population with high prevalence of dermatophytic onychomycosis or non-dermatophytic onychomycosis (e.g., a population of soldiers, long-distance runners, or miners).

In methods of treating or preventing onychomycosis in a subject, the onychomycosis may be caused by a fungus in the genus Trichophyton (e.g., Trichophyton rubrum, Trichophyton mentagrophytes). In some embodiments, the invention provides methods of treating or preventing onychomycosis caused by Trichophyton rubrum in a subject by administering (e.g., subcutaneously administering) to the subject CD101 or a CD101 analog in salt or neutral form.

In methods of treating or preventing onychomycosis in a subject, the onychomycosis may be caused by a fungus in the genus Candida (e.g., Candida albicans, C. glabrata, C. dubliniensis, C. krusei, C. parapsilosis, C. tropicalis, C. orthopsilosis, C. guilliermondii, C. rugosa, and C. lusitaniae). In some embodiments, the invention provides methods of treating or preventing onychomycosis caused by Candida albicans in a subject by administering (e.g., subcutaneously administering) to the subject CD101 or a CD101 analog in salt or neutral form.

In some embodiments of the methods described herein, the administering step includes administering (e.g., subcutaneously administering) doses of about 25 mg to about 600 mg of CD101 or a CD101 analog in salt or neutral form thereof, in which one or more doses are administered over a period of 4 to 52 weeks and in which no more than one dose is administered per week. In some embodiments, one dose of CD101 or a CD101 analog in salt or neutral form is administered (e.g., subcutaneously administered) once every 4 weeks for 4 to 52 weeks (e.g., 4 to 48, 4 to 44, 4 to 40, 4 to 36, 4 to 32, 4 to 28, 4 to 24, 4 to 20, 4 to 16, 4 to 12, or 4 to 8 weeks; 4, 8, 12, 16, 20, 24, 28, 32, 36, 40, 44, 48, or 52 weeks). In some embodiments, one dose of CD101 or a CD101 analog in salt or neutral form is administered (e.g., subcutaneously administered) once every 3 weeks for 6 to 51 weeks (e.g., 6 to 48, 6 to 45, 6 to 42, 6 to 39, 6 to 36, 6 to 33, 6 to 30, 6 to 27, 6 to 24, 6 to 21, 6 to 18, 6 to 15, 6 to 12, or 6 to 9 weeks; 6, 9, 12, 15, 18, 21, 24, 27, 30, 33, 36, 39, 42, 45, 48, or 51 weeks). In some embodiments, one dose of CD101 or a CD101 analog in salt or neutral form is administered (e.g., subcutaneously administered) once every 2 weeks for 4 to 52 weeks (e.g., 4 to 50, 4 to 48, 4 to 46, 4 to 44, 4 to 42, 4 to 40, 4 to 38, 4 to 36, 4 to 34, 4 to 32, 4 to 30, 4 to 28, 4 to 26, 4 to 24, 4 to 22, 4 to 20, 4 to 18, 4 to 16, 4 to 14, 4 to 12, 4 to 10, 4 to 8, or 4 to 6 weeks; 2, 4, 6, 8, 10, 12, 14, 16, 18, 20, 22, 24, 26, 28, 30, 32, 34, 36, 38, 40, 42, 44, 46, 48, 50, or 52 weeks). In some embodiments, one dose of CD101 or a CD101 analog in salt or neutral form is administered (e.g., subcutaneously administered) once every week for 4 to 52 weeks (e.g., 4 to 51, 4 to 50, 4 to 49, 4 to 48, 4 to 47, 4 to 46, 4 to 45, 4 to 44, 4 to 43, 4 to 42, 4 to 41, 4 to 40, 4 to 39, 4 to 38, 4 to 37, 4 to 36, 4 to 35, 4 to 34, 4 to 33, 4 to 32, 4 to 31, 4 to 30, 4 to 29, 4 to 28, 4 to 27, 4 to 26, 4 to 25, 4 to 24, 4 to 23, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, or 4 to 5 weeks; 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks).

In some embodiments of the methods described herein, the administering step includes administered (e.g., subcutaneously administered) doses of about 25 mg to about 600 mg of CD101 or a CD101 analog in salt or neutral form thereof, in which three or more doses are administered over a period of 12 to 24 weeks and in which no more than one dose is administered per week. In some embodiments, one dose of CD101 or a CD101 analog in salt or neutral form is administered (e.g., subcutaneously administered) once every 4 weeks for 12 to 24 weeks (e.g., 12 to 20 or 12 to 16 weeks; 12, 16, 20, or 24 weeks). In some embodiments, one dose of CD101 or a CD101 analog in salt or neutral form is administered (e.g., subcutaneously administered) once every 3 weeks for 12 to 24 weeks (e.g., 12 to 21, 12 to 18, or 12 to 15 weeks; 12, 15, 18, 21, or 24 weeks). In some embodiments, one dose of CD101 or a CD101 analog in salt or neutral form is administered (e.g., subcutaneously administered) once every 2 weeks for 12 to 24 weeks (e.g., 12 to 22, 12 to 20, 12 to 18, 12 to 16, or 12 to 14 weeks; 12, 14, 16, 18, 20, 22, or 24 weeks). In some embodiments, one dose of CD101 or a CD101 analog in salt or neutral form is administered (e.g., subcutaneously administered) once every week for 12 to 24 weeks (e.g., 12 to 23, 12 to 22, 12 to 21, 12 to 20, 12 to 19, 12 to 18, 12 to 17, 12 to 16, 12 to 15, 12 to 14, or 12 to 13 weeks; 12, 13, 14, 15, 16, 17, 18, 19, 20, 21, 22, 23, or 24 weeks).

In some embodiments of the methods described herein, the administering step includes subcutaneously administering CD101 or a CD101 analog in salt or neutral form in combination with a human hyaluronidase enzyme, e.g., rHuPH20, e.g., PEGylated rHuPH20, e.g., HALOZYME ENHANZE® as disclosed in U.S. Patent Publication 2017/0290796 and U.S. Pat. Nos. 9,211,315, 9,034,323, 9,333,244, 8,343,487, and 7,767,429, each of which is incorporated herein by reference.

Human hyaluronidase enzymes, e.g., rHuPH20, are able to break down hyaluronan, a naturally occurring glycosaminoglycan biopolymer found in connective tissues, e.g., cartilage, and epithelial cells which due to its viscosity can reduce uptake of subcutaneously administered therapeutic compounds. The breakdown of hyaluronan in the body enables therapeutic molecules that are administered subcutaneously to be adequately dispersed and absorbed at the local treatment site. Human hyaluronidase enzymes may be administered intravenously, increasing the length of time that the enzyme is in the body. Alternatively, the enzyme may be administered subcutaneously or topically to a localized area of the body requiring treatment. The CD101 or a CD101 analog in salt or neutral form may be co-administered in a formulation with a human hyaluronidase enzyme. Alternatively, the CD101 or a CD101 analog in salt or neutral form may be administered separately from the human hyaluronidase enzyme. In some embodiments, the human hyaluronidase enzyme is administered prior to (e.g. at least 1 minute, 1 hour, 1 day, or 1 week) the administration of CD101 or a CD101 analog in salt or neutral form.

The invention also features methods of killing a Trichophyton, Microsporum, or Epidermophyton by exposing the Trichophyton, Microsporum, or Epidermophyton to CD101 or a CD101 analog in salt or neutral form, in an amount and for a duration sufficient to kill the Trichophyton, Microsporum, or Epidermophyton. In some embodiments, Trichophyton is Trichophyton rubrum or Trichophyton interdigitale. In some embodiments, Microsporum is Microsporum canis or Microsporum audouinii. In some embodiments, Epidermophyton is Epidermophyton floccosum.

### VI. Pharmaceutical Compositions and Preparations

CD101 or a CD101 analog may be prepared in a pharmaceutical composition. In some embodiments, the pharmaceutical composition includes a salt of CD101 or a CD101 analog, or a neutral form thereof, and pharmaceutically acceptable carriers and excipients. Depending on the mode of administration (e.g., subcutaneously, topically, orally, intravenously, intravaginally, intraorally, intramuscularly, intradermally, intraarterially, or by inhalation) and the dosage, CD101 or a CD101 analog used in the methods described herein may be formulated into suitable pharmaceutical compositions to permit facile delivery. In some embodiments, for subcutaneous administration of CD101 or a CD101 analog in salt or neutral form in methods described herein, CD101 or a CD101 analog may be formulated as an aqueous pharmaceutical composition, e.g., an aqueous pharmaceutical composition at a pH of from 4 to 8. In some embodiments, CD101 or a CD101 analog in salt form may be formulated in a pharmaceutical composition as CD101 acetate or a CD101 analog acetate. A summary of such techniques is found in Remington: The Science and Practice of Pharmacy, 22^{nd} Edition, Lippincott Williams & Wilkins, (2012); and Encyclopedia of Pharmaceutical Technology, eds. J. Swarbrick and J. C. Boylan, 2006, Marcel Dekker, New York, each of which is incorporated herein by reference.

In some embodiments of the methods described herein, when CD101 or a CD101 analog in salt or neutral form and at least one antifungal agent are used in combination (e.g., used in combination by administering substantially simultaneously or used in combination separately), CD101, or a CD101 analog, and the antifungal agent(s) may be formulated in separate pharmaceutical compositions. In some embodiments, CD101, or a CD101 analog, and the antifungal agent(s) may be formulated in the same pharmaceutical composition.

CD101 or a CD101 analog or pharmaceutical compositions containing CD101 or a CD101 analog may be administered in a manner compatible with the dosage formulation and in such amount as is therapeutically effective to result in an improvement or remediation of the symptoms. The pharmaceutical compositions are administered in a variety of dosage forms, e.g., subcutaneous dosage forms, topical dosage forms, intravenous dosage forms, and oral dosage forms (e.g., ingestible solutions, drug release capsules). CD101 or a CD101 analog or pharmaceutical compositions containing CD101 or a CD101 analog may be in the form of, e.g., tablets, capsules, pills, powders, granulates, suspensions, emulsions, solutions, gels including hydrogels, pastes, ointments, creams, plasters, drenches, osmotic delivery devices, suppositories, enemyas, injectables, implants, sprays, preparations suitable for iontophoretic delivery, or aerosols. The compositions may be formulated according to conventional pharmaceutical practice.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol.

The pharmaceutical compositions can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Gibson (ed.) Pharmaceutical Preformulation and Formulation (2^{nd} ed.) Taylor & Francis Group, CRC Press (2009).

The pharmaceutical compositions can be prepared in the form of an oral formulation. Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

The pharmaceutical composition may be formed in a unit dose form as needed. The amount of active component, e.g., CD101, included in the pharmaceutical compositions are such that a suitable dose within the designated range is provided (e.g., a dose within the range of 0.01-100 mg/kg of body weight).

### VII. Routes, Dosage, and Administration

CD101, a CD101 analog, or pharmaceutical compositions including CD101 or a CD101 analog may be formulated for, e.g., subcutaneous administration, topical administration, oral administration, intravenous administration, intravaginal administration, intraoral administration, intramuscular administration, intradermal administration, intraarterial administration, or by inhalation. In particular embodiments, CD101, a CD101 analog, or pharmaceutical compositions including CD101 or a CD101 analog may be formulated for subcutaneous administration. In particular embodiments, CD101, a CD101 analog, or pharmaceutical compositions including CD101 or a CD101 analog may be formulated for subcutaneous administration in the treatment or prevention of onychomycosis. For injectable formulations, various effective pharmaceutical carriers are known in the art. See, e.g., Remington: The Science and Practice of Pharmacy, 22nd ed., (2012) and ASHP Handbook on Injectable Drugs, 18th ed., (2014).

The dosage of CD101, a CD101 analog, or the pharmaceutical composition including CD101 or a CD101 analog depends on factors including the route of administration, the infection to be treated, and physical characteristics, e.g., age, weight, general health, of the subject (e.g., a human). The dosage may be adapted by the physician in accordance with conventional factors such as the extent of the disease and different parameters of the subject. Typically, the amount of CD101, a CD101 analog, or the pharmaceutical composition contained within a single dose may be an amount that effectively treats the infection without inducing significant toxicity.

In some embodiments of the methods described herein, the administering step includes administering (e.g., subcutaneously administering) doses of about 25 mg to about 600 mg (e.g., about 25 mg to about 550 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 190 mg, about 25 mg to about 180 mg, about 25 mg to about 170 mg, about 25 mg to about 160 mg, about 25 mg to about 150 mg, about 25 mg to about 140 mg, about 25 mg to about 130 mg, about 25 mg to about 120 mg, about 25 mg to about 110 mg, about 25 mg to about 100 mg, about 25 mg to about 90 mg, about 25 mg to about 80 mg, about 25 mg to about 70 mg, about 25 mg to about 60 mg, about 25 mg to about 50 mg, about 25 mg to about 40 mg, about 25 mg to about 30 mg, about 50 mg to about 600 mg, about 100 mg to about 600 mg, about 150 mg to about 600 mg, about 200 mg to about 600 mg, about 250 mg to about 600 mg, about 300 mg to about 600 mg, about 350 mg to about 600 mg, about 400 mg to about 600 mg, about 450 mg to about 600 mg, about 500 mg to about 600 mg, about 550 mg to about 600 mg, about 50 mg to about 550 mg, about 100 mg to about 500 mg, about 150 mg to about 450 mg, about 200 mg to about 400 mg, about 250 mg to about 350 mg, about 250 mg to about 300 mg, about 300 mg to about 350 mg) of CD101 or a CD101 analog in salt or neutral form thereof, in which one or more doses are administered over a period of 4 to 52 weeks and in which no more than one dose is administered per week.

In some embodiments of the methods described herein, the administering step includes administering (e.g., subcutaneously administering) doses of about 25 mg to about 600 mg (e.g., about 25 mg to about 550 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 190 mg, about 25 mg to about 180 mg, about 25 mg to about 170 mg, about 25 mg to about 160 mg, about 25 mg to about 150 mg, about 25 mg to about 140 mg, about 25 mg to about 130 mg, about 25 mg to about 120 mg, about 25 mg to about 110 mg, about 25 mg to about 100 mg, about 25 mg to about 90 mg, about 25 mg to about 80 mg, about 25 mg to about 70 mg, about 25 mg to about 60 mg, about 25 mg to about 50 mg, about 25 mg to about 40 mg, about 25 mg to about 30 mg, about 50 mg to about 600 mg, about 100 mg to about 600 mg, about 150 mg to about 600 mg, about 200 mg to about 600 mg, about 250 mg to about 600 mg, about 300 mg to about 600 mg, about 350 mg to about 600 mg, about 400 mg to about 600 mg, about 450 mg to about 600 mg, about 500 mg to about 600 mg, about 550 mg to about 600 mg, about 50 mg to about 550 mg, about 100 mg to about 500 mg, about 150 mg to about 450 mg, about 200 mg to about 400 mg, about 250 mg to about 350 mg, about 250 mg to about 300 mg, about 300 mg to about 350 mg) of CD101 or a CD101 analog in salt or neutral form thereof, in which one or more doses are administered over a period of 1 to 4 weeks (e.g., 1, 2, 3, or 4 weeks) and in which no more than one dose is administered per week.

In some embodiments, one dose of CD101 or a CD101 analog in salt or neutral form is administered (e.g., subcutaneously administered) once every 4 weeks for 4 to 52 weeks (e.g., 4 to 48, 4 to 44, 4 to 40, 4 to 36, 4 to 32, 4 to 28, 4 to 24, 4 to 20, 4 to 16, 4 to 12, or 4 to 8 weeks; 4, 8, 12, 16, 20, 24, 28, 32, 36, 40, 44, 48, or 52 weeks). In some embodiments, one dose of CD101 or a CD101 analog in salt or neutral form is administered (e.g., subcutaneously administered) once every 3 weeks for 6 to 51 weeks (e.g., 6 to 48, 6 to 45, 6 to 42, 6 to 39, 6 to 36, 6 to 33, 6 to 30, 6 to 27, 6 to 24, 6 to 21, 6 to 18, 6 to 15, 6 to 12, or 6 to 9 weeks; 6, 9, 12, 15, 18, 21, 24, 27, 30, 33, 36, 39, 42, 45, 48, or 51 weeks). In some embodiments, one dose of CD101 or a CD101 analog in salt or neutral form is administered (e.g., subcutaneously administered) once every 2 weeks for 4 to 52 weeks (e.g., 4 to 50, 4 to 48, 4 to 46, 4 to 44, 4 to 42, 4 to 40, 4 to 38, 4 to 36, 4 to 34, 4 to 32, 4 to 30, 4 to 28, 4 to 26, 4 to 24, 4 to 22, 4 to 20, 4 to 18, 4 to 16, 4 to 14, 4 to 12, 4 to 10, 4 to 8, or 4 to 6 weeks; 2, 4, 6, 8, 10, 12, 14, 16, 18, 20, 22, 24, 26, 28, 30, 32, 34, 36, 38, 40, 42, 44, 46, 48, 50, or 52 weeks). In some embodiments, one dose of CD101 or a CD101 analog in salt or neutral form is administered (e.g., subcutaneously administered) once every week for 4 to 52 weeks (e.g., 4 to 51, 4 to 50, 4 to 49, 4 to 48, 4 to 47, 4 to 46, 4 to 45, 4 to 44, 4 to 43, 4 to 42, 4 to 41, 4 to 40, 4 to 39, 4 to 38, 4 to 37, 4 to 36, 4 to 35, 4 to 34, 4 to 33, 4 to 32, 4 to 31, 4 to 30, 4 to 29, 4 to 28, 4 to 27, 4 to 26, 4 to 25, 4 to 24, 4 to 23, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, or 4 to 5 weeks; 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks). In some embodiments, one dose of CD101 or a CD101 analog in salt or neutral form is administered (e.g., subcutaneously administered) once for 1 to 4 weeks (e.g., 1, 2, 3, or 4 weeks).

In some embodiments of the methods described herein, the administering step includes administered (e.g., subcutaneously administered) doses of about 25 mg to about 600 mg (e.g., about 25 mg to about 550 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 190 mg, about 25 mg to about 180 mg, about 25 mg to about 170 mg, about 25 mg to about 160 mg, about 25 mg to about 150 mg, about 25 mg to about 140 mg, about 25 mg to about 130 mg, about 25 mg to about 120 mg, about 25 mg to about 110 mg, about 25 mg to about 100 mg, about 25 mg to about 90 mg, about 25 mg to about 80 mg, about 25 mg to about 70 mg, about 25 mg to about 60 mg, about 25 mg to about 50 mg, about 25 mg to about 40 mg, about 25 mg to about 30 mg, about 50 mg to about 600 mg, about 100 mg to about 600 mg, about 150 mg to about 600 mg, about 200 mg to about 600 mg, about 250 mg to about 600 mg, about 300 mg to about 600 mg, about 350 mg to about 600 mg, about 400 mg to about 600 mg, about 450 mg to about 600 mg, about 500 mg to about 600 mg, about 550 mg to about 600 mg, about 50 mg to about 550 mg, about 100 mg to about 500 mg, about 150 mg to about 450 mg, about 200 mg to about 400 mg, about 250 mg to about 350 mg, about 250 mg to about 300 mg, about 300 mg to about 350 mg) of CD101 or a CD101 analog in salt or neutral form thereof, in which three or more doses are administered over a period of 12 to 24 weeks and in which no more than one dose is administered per week.

In some embodiments, one dose of CD101 or a CD101 analog in salt or neutral form is administered (e.g., subcutaneously administered) once every 4 weeks for 12 to 24 weeks (e.g., 12 to 20 or 12 to 16 weeks; 12, 16, 20, or 24 weeks). In some embodiments, one dose of CD101 or a CD101 analog in salt or neutral form is administered (e.g., subcutaneously administered) once every 3 weeks for 12 to 24 weeks (e.g., 12 to 21, 12 to 18, or 12 to 15 weeks; 12, 15, 18, 21, or 24 weeks). In some embodiments, one dose of CD101 or a CD101 analog in salt or neutral form is administered (e.g., subcutaneously administered) once every 2 weeks for 12 to 24 weeks (e.g., 12 to 22, 12 to 20, 12 to 18, 12 to 16, or 12 to 14 weeks; 12, 14, 16, 18, 20, 22, or 24 weeks). In some embodiments, one dose of CD101 or a CD101 analog in salt or neutral form is administered (e.g., subcutaneously administered) once every week for 12 to 24 weeks (e.g., 12 to 23, 12 to 22, 12 to 21, 12 to 20, 12 to 19, 12 to 18, 12 to 17, 12 to 16, 12 to 15, 12 to 14, or 12 to 13 weeks; 12, 13, 14, 15, 16, 17, 18, 19, 20, 21, 22, 23, or 24 weeks).

In some embodiments of the methods described herein, the administering step includes administered (e.g., subcutaneously administered) doses of about 25 mg to about 600 mg (e.g., about 25 mg to about 550 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 190 mg, about 25 mg to about 180 mg, about 25 mg to about 170 mg, about 25 mg to about 160 mg, about 25 mg to about 150 mg, about 25 mg to about 140 mg, about 25 mg to about 130 mg, about 25 mg to about 120 mg, about 25 mg to about 110 mg, about 25 mg to about 100 mg, about 25 mg to about 90 mg, about 25 mg to about 80 mg, about 25 mg to about 70 mg, about 25 mg to about 60 mg, about 25 mg to about 50 mg, about 25 mg to about 40 mg, about 25 mg to about 30 mg, about 50 mg to about 600 mg, about 100 mg to about 600 mg, about 150 mg to about 600 mg, about 200 mg to about 600 mg, about 250 mg to about 600 mg, about 300 mg to about 600 mg, about 350 mg to about 600 mg, about 400 mg to about 600 mg, about 450 mg to about 600 mg, about 500 mg to about 600 mg, about 550 mg to about 600 mg, about 50 mg to about 550 mg, about 100 mg to about 500 mg, about 150 mg to about 450 mg, about 200 mg to about 400 mg, about 250 mg to about 350 mg, about 250 mg to about 300 mg, about 300 mg to about 350 mg) of CD101 or a CD101 analog in salt or neutral form thereof, in which three or more doses are administered over a period of 12 weeks and in which no more than one dose is administered per week.

In some embodiments, three doses of CD101 or a CD101 analog in salt or neutral form is administered over a period of 12 weeks (e.g., one dose every 4 weeks). In some embodiments, four doses of CD101 or a CD101 analog in salt or neutral form is administered over a period of 12 weeks (e.g., one dose every 3 weeks). In some embodiments, six doses of CD101 or a CD101 analog in salt or neutral form is administered over a period of 12 weeks (e.g., one dose every 2 weeks). In some embodiments, 12 doses of CD101 or a CD101 analog in salt or neutral form is administered over a period of 12 weeks (e.g., one dose every week).

In some embodiments of the methods described herein, the administering step includes administered (e.g., subcutaneously administered) doses of about 25 mg to about 600 mg (e.g., about 25 mg to about 550 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 190 mg, about 25 mg to about 180 mg, about 25 mg to about 170 mg, about 25 mg to about 160 mg, about 25 mg to about 150 mg, about 25 mg to about 140 mg, about 25 mg to about 130 mg, about 25 mg to about 120 mg, about 25 mg to about 110 mg, about 25 mg to about 100 mg, about 25 mg to about 90 mg, about 25 mg to about 80 mg, about 25 mg to about 70 mg, about 25 mg to about 60 mg, about 25 mg to about 50 mg, about 25 mg to about 40 mg, about 25 mg to about 30 mg, about 50 mg to about 600 mg, about 100 mg to about 600 mg, about 150 mg to about 600 mg, about 200 mg to about 600 mg, about 250 mg to about 600 mg, about 300 mg to about 600 mg, about 350 mg to about 600 mg, about 400 mg to about 600 mg, about 450 mg to about 600 mg, about 500 mg to about 600 mg, about 550 mg to about 600 mg, about 50 mg to about 550 mg, about 100 mg to about 500 mg, about 150 mg to about 450 mg, about 200 mg to about 400 mg, about 250 mg to about 350 mg, about 250 mg to about 300 mg, about 300 mg to about 350 mg) of CD101 or a CD101 analog in salt or neutral form thereof, in which four or more doses are administered over a period of 16 weeks and in which no more than one dose is administered per week.

In some embodiments, four doses of CD101 or a CD101 analog in salt or neutral form is administered over a period of 16 weeks (e.g., one dose every 4 weeks). In some embodiments, five doses of CD101 or a CD101 analog in salt or neutral form is administered over a period of 16 weeks (e.g., one dose every 3 weeks). In some embodiments, eight doses of CD101 or a CD101 analog in salt or neutral form is administered over a period of 16 weeks (e.g., one dose every 2 weeks). In some embodiments, 16 doses of CD101 or a CD101 analog in salt or neutral form is administered over a period of 16 weeks (e.g., one dose every week).

In some embodiments of the methods described herein, the administering step includes administered (e.g., subcutaneously administered) doses of about 25 mg to about 600 mg (e.g., about 25 mg to about 550 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 190 mg, about 25 mg to about 180 mg, about 25 mg to about 170 mg, about 25 mg to about 160 mg, about 25 mg to about 150 mg, about 25 mg to about 140 mg, about 25 mg to about 130 mg, about 25 mg to about 120 mg, about 25 mg to about 110 mg, about 25 mg to about 100 mg, about 25 mg to about 90 mg, about 25 mg to about 80 mg, about 25 mg to about 70 mg, about 25 mg to about 60 mg, about 25 mg to about 50 mg, about 25 mg to about 40 mg, about 25 mg to about 30 mg, about 50 mg to about 600 mg, about 100 mg to about 600 mg, about 150 mg to about 600 mg, about 200 mg to about 600 mg, about 250 mg to about 600 mg, about 300 mg to about 600 mg, about 350 mg to about 600 mg, about 400 mg to about 600 mg, about 450 mg to about 600 mg, about 500 mg to about 600 mg, about 550 mg to about 600 mg, about 50 mg to about 550 mg, about 100 mg to about 500 mg, about 150 mg to about 450 mg, about 200 mg to about 400 mg, about 250 mg to about 350 mg, about 250 mg to about 300 mg, about 300 mg to about 350 mg) of CD101 or a CD101 analog in salt or neutral form thereof, in which five or more doses are administered over a period of 20 weeks and in which no more than one dose is administered per week.

In some embodiments, five doses of CD101 or a CD101 analog in salt or neutral form is administered over a period of 20 weeks (e.g., one dose every 4 weeks). In some embodiments, six doses of CD101 or a CD101 analog in salt or neutral form is administered over a period of 20 weeks (e.g., one dose every 3 weeks). In some embodiments, ten doses of CD101 or a CD101 analog in salt or neutral form is administered over a period of 20 weeks (e.g., one dose every 2 weeks). In some embodiments, 20 doses of CD101 or a CD101 analog in salt or neutral form is administered over a period of 20 weeks (e.g., one dose every week).

In some embodiments of the methods described herein, the administering step includes administered (e.g., subcutaneously administered) doses of about 25 mg to about 600 mg (e.g., about 25 mg to about 550 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 190 mg, about 25 mg to about 180 mg, about 25 mg to about 170 mg, about 25 mg to about 160 mg, about 25 mg to about 150 mg, about 25 mg to about 140 mg, about 25 mg to about 130 mg, about 25 mg to about 120 mg, about 25 mg to about 110 mg, about 25 mg to about 100 mg, about 25 mg to about 90 mg, about 25 mg to about 80 mg, about 25 mg to about 70 mg, about 25 mg to about 60 mg, about 25 mg to about 50 mg, about 25 mg to about 40 mg, about 25 mg to about 30 mg, about 50 mg to about 600 mg, about 100 mg to about 600 mg, about 150 mg to about 600 mg, about 200 mg to about 600 mg, about 250 mg to about 600 mg, about 300 mg to about 600 mg, about 350 mg to about 600 mg, about 400 mg to about 600 mg, about 450 mg to about 600 mg, about 500 mg to about 600 mg, about 550 mg to about 600 mg, about 50 mg to about 550 mg, about 100 mg to about 500 mg, about 150 mg to about 450 mg, about 200 mg to about 400 mg, about 250 mg to about 350 mg, about 250 mg to about 300 mg, about 300 mg to about 350 mg) of CD101 or a CD101 analog in salt or neutral form thereof, in which six or more doses are administered over a period of 24 weeks and in which no more than one dose is administered per week.

In some embodiments, six doses of CD101 or a CD101 analog in salt or neutral form is administered over a period of 24 weeks (e.g., one dose every 4 weeks). In some embodiments, eight doses of CD101 or a CD101 analog in salt or neutral form is administered over a period of 24 weeks (e.g., one dose every 3 weeks). In some embodiments, 12 doses of CD101 or a CD101 analog in salt or neutral form is administered over a period of 24 weeks (e.g., one dose every 2 weeks). In some embodiments, 24 doses of CD101 or a CD101 analog in salt or neutral form is administered over a period of 24 weeks (e.g., one dose every week).

In some embodiments of the methods described herein, the administering step includes administered (e.g., subcutaneously administered) doses of about 25 mg to about 600 mg (e.g., about 25 mg to about 550 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 190 mg, about 25 mg to about 180 mg, about 25 mg to about 170 mg, about 25 mg to about 160 mg, about 25 mg to about 150 mg, about 25 mg to about 140 mg, about 25 mg to about 130 mg, about 25 mg to about 120 mg, about 25 mg to about 110 mg, about 25 mg to about 100 mg, about 25 mg to about 90 mg, about 25 mg to about 80 mg, about 25 mg to about 70 mg, about 25 mg to about 60 mg, about 25 mg to about 50 mg, about 25 mg to about 40 mg, about 25 mg to about 30 mg, about 50 mg to about 600 mg, about 100 mg to about 600 mg, about 150 mg to about 600 mg, about 200 mg to about 600 mg, about 250 mg to about 600 mg, about 300 mg to about 600 mg, about 350 mg to about 600 mg, about 400 mg to about 600 mg, about 450 mg to about 600 mg, about 500 mg to about 600 mg, about 550 mg to about 600 mg, about 50 mg to about 550 mg, about 100 mg to about 500 mg, about 150 mg to about 450 mg, about 200 mg to about 400 mg, about 250 mg to about 350 mg, about 250 mg to about 300 mg, about 300 mg to about 350 mg) of CD101 or a CD101 analog in salt or neutral form thereof, in which 3 to 24 doses (e.g., 3 to 23, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4 doses) are administered over a period of 12 to 24 weeks (e.g., 12 to 23, 12 to 22, 12 to 21, 12 to 20, 12 to 19, 12 to 18, 12 to 17, 12 to 16, 12 to 15, 12 to 14, or 12 to 13 weeks) and in which no more than one dose is administered per week.

In some embodiments of the methods described herein, the administering step includes administered (e.g., subcutaneously administered) doses of about 25 mg to about 600 mg (e.g., about 25 mg to about 550 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 190 mg, about 25 mg to about 180 mg, about 25 mg to about 170 mg, about 25 mg to about 160 mg, about 25 mg to about 150 mg, about 25 mg to about 140 mg, about 25 mg to about 130 mg, about 25 mg to about 120 mg, about 25 mg to about 110 mg, about 25 mg to about 100 mg, about 25 mg to about 90 mg, about 25 mg to about 80 mg, about 25 mg to about 70 mg, about 25 mg to about 60 mg, about 25 mg to about 50 mg, about 25 mg to about 40 mg, about 25 mg to about 30 mg, about 50 mg to about 600 mg, about 100 mg to about 600 mg, about 150 mg to about 600 mg, about 200 mg to about 600 mg, about 250 mg to about 600 mg, about 300 mg to about 600 mg, about 350 mg to about 600 mg, about 400 mg to about 600 mg, about 450 mg to about 600 mg, about 500 mg to about 600 mg, about 550 mg to about 600 mg, about 50 mg to about 550 mg, about 100 mg to about 500 mg, about 150 mg to about 450 mg, about 200 mg to about 400 mg, about 250 mg to about 350 mg, about 250 mg to about 300 mg, about 300 mg to about 350 mg) of CD101 or a CD101 analog in salt or neutral form thereof, in which 6 to 12 doses (e.g., 6 to 11, 6 to 10, 6 to 9, 6 to 8, or 6 to 7 doses) are administered over a period of 12 to 24 weeks (e.g., 12 to 23, 12 to 22, 12 to 21, 12 to 20, 12 to 19, 12 to 18, 12 to 17, 12 to 16, 12 to 15, 12 to 14, or 12 to 13 weeks) and in which no more than one dose is administered per week.

As used herein, the amount in each dose refers to the amount of CD101 or a CD101 analog that does not include the negative counterion (e.g., an acetate) if CD101 or the CD101 analog is in its salt form. For example, a dose of about 400 mg or 200 mg of CD101 in salt or neutral form refers to 400 mg or 200 mg of CD101 or a CD101 analog, not including the acetate ion if CD101 or the CD101 analog is in an acetate salt form.

The following Examples are intended to further illustrate but not to limit the disclosure herein.

### EXAMPLES

### Example 1. Efficacy of CD101 in the Treatment of Trichophyton mentagrophytes Dermatophytosis in a Guinea Pig Model

Animal Care and Use Statement

All procedures in the protocol were in compliance with the Animal Welfare Act, the Guide for the Care and Use of Laboratory Animals, and the Office of Laboratory Animal Welfare. Guinea pigs were used under an approved protocol by the Institutional Animal Care and Use Committee.

Procurement and Housing of Animals

A request was made to the Animal Resource Center (ARC) at Case Western Reserve University to order the required number of male albino Harlan-Sprague-Dawley Guinea-Pigs (San Diego, Calif.) with a body weight of 450-500 g. Animals were allowed to acclimate for a minimum of 5 days prior to use. Environmental controls for the animal room were set to maintain a temperature of 16 to 22° C., a relative humidity of 30-70%, and a 12:12 light-dark cycle.

Preparation of Standard Inoculum

a. Organism

T. mentagrophytes ATCC 24953 was used as the infecting fungus. This strain was selected because it can cause skin infection and results in inflammatory reaction.

b. Supplies and Equipment

hemacytometer, vortex, sterile conical tubes, centrifuge, serological pipettes, gloves, disposable syringes, cell counter, adjustable volume pipettes, culture plates, microscope, shaking water bath

c. Reagents and Media

sterile normal saline (0.85%), chloramphenicol (0.05 g/L of agar medium), potato dextrose agar (Difco Laboratories), sabouraud Dextrose Agar (Difco Laboratories)

d. Procedure

T. mentagrophytes (from frozen stock) was sub-cultured on Potato Dextrose Agar plates and incubated at 30° C. for 5-7 days. The colonies were scraped from the plates using sterile normal saline (NaCl 0.85%) solution. After washing for three times with sterile saline the conidia were re-suspended in the same solution. Ten-fold dilutions of conidia suspension were prepared and counted using a hemacytometer. Working suspension of conidia was prepared at a final concentration of 1×10⁷ Colony Forming Units (CFUs)/100 μL in normal saline.

e. Verification of Inoculum Count

To check the inoculum count, ten-fold dilutions of T. mentagrophytes working conidial suspension were plated onto Sabouraud Dextrose Agar media. The plates were incubated at 30° C. for 3-4 days and the CFUs determined.

Infection

Each animal was anesthetized with a cocktail of xylazine and ketamine: 0.2 mL intramuscularly. Using an electric shaver, hair was clipped on left side of the guinea pig back. A closer shave was done with a disposable razor. Using a stencil, a square of 2.5 cm×2.5 cm was marked. The marked skin area was abraded with sterile fine grit sandpaper. A cell suspension containing 1×10⁷ conidia in 100 μL was applied and rubbed thoroughly on the abraded skin.

Test Compound

CD101 and the vehicle control for the study.

Treatment Groups

Infected guinea pigs were randomized into the following groups (5 per group): CD101, 35 mg/kg daily, terbinafine 10 mg/kg (as a positive control), and a vehicle control. Two additional groups were studied by dosing once weekly, CD101 at 35 mg/kg and terbinafine at 10 mg/kg.

Schedule of Treatment

Beginning two hours post inoculation, animals were treated subcutaneously (SQ) for CD101 and orally (PO) terbinafine once daily for a period of nine days, or once a week on days 1 and 8.

a. Study Diagram (schedule)

b. Study Procedure

Study Day 1: Infected the animals with T. mentagrophytes and begin treatments.

Study Day 9 (treatment day 9): End treatments.

Study Day 10: Clinical evaluation of the four quadrants in the marked area was done. Hair samples were obtained from each quadrant (10 per quadrant). Hairs were planted in Potato Dextrose Agar, and incubated for 48 h for mycological evaluation. Procedures for clinical and mycological evaluations are described below.

c. Test Article Administration

CD101 was administered SQ in 0.3 mL, while the terbinafine was given orally in 0.3 mL. Vehicle treated group was also included.

Hair Root Invasion Test for Mycological Evaluation

Hair samples were removed with a sterile forceps from four quadrants. Ten hair samples from each quadrant were inoculated onto the corresponding quadrant on the Potato Dextrose agar plate and incubated at 30° C. for 2 days. The fungal growth at the hair root was examined under a stereomicroscope. The effectiveness of a compound in reducing the number of mycologically positive hair sample per treated group is expressed as percentage relative to the vehicle control group of animals using the following formula:

% Efficacy=100−(T×100/K)

where, T=Positive hair in the test group, K=Positive hair in the Untreated control.

Clinical Evaluation

Clinical assessment of local changes of the infected skin area was scored as follows:

0=No lesions

1=Few slightly erythematous places on the skin

2=Well-defined redness, swelling with bristling hairs.

3=Large areas or marked redness incrustation, scaling, bald patches, ulcerated in places.

4=Partial damage to the integument and loss of hair

5=Extensive damage to the integument and complete loss of hair at the site of infection.

Any other local skin reactions were noted. The assessment of clinical evaluation in the change of scores per treatment group is expressed as percentage relative to the vehicle control group of animals using the following formula:

% Efficacy=100−(T×100/K)

where, T=Scores in the test group. K=Scores in the untreated control

Statistical Analysis

A statistical analysis of data from the clinical and mycological studies was performed. Significance was determined using test(s) as appropriate. The treated groups were compared to the vehicle-treated control to determine antifungal activity.

Results

Clinical Efficacy.

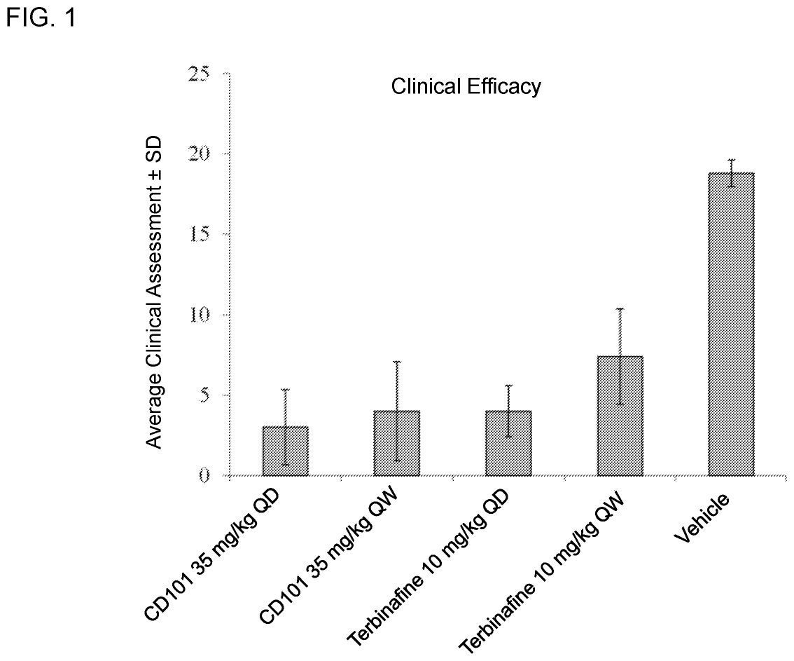

Table 3 and FIG. 1 show the clinical efficacy of each test compound as compared to the vehicle control. As expected, the vehicle control guinea pigs showed hair loss and ulcerated, scaly skin. Percent efficacies for CD101 35 mg/kg daily and CD101 35 mg/kg once a week were 84.0 and 78.7, respectively. Percent efficacies for terbinafine 10 mg/kg daily and terbinafine 10 mg/kg once a week were 78.7 and 60.6, respectively. All treatment groups showed significant efficacy when compared to the vehicle control (P-values of <0.001). There was no significant difference between treatment groups.

Animals treated with CD101 daily and weekly showed fewer clinical signs than the vehicle control group beginning on day 5 post inoculation. Also, no bruising or redness was noted at the injection site for the CD101-treated animals.

Mycological Efficacy.

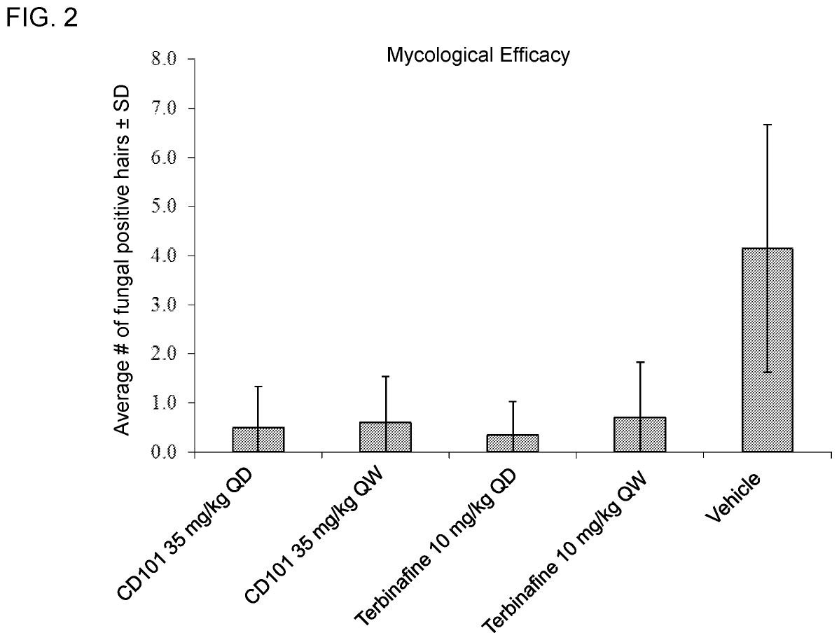

Table 3 and FIG. 2 show the mycological efficacy of each test compound as compared to the vehicle control. The vehicle control behaved as expected having the highest average fungal positive hairs. Percent efficacies for CD101 35 mg/kg daily and CD101 35 mg/kg once a week were 88.0 and 85.5, respectively. Percent efficacies for terbinafine 10 mg/kg daily and terbinafine 10 mg/kg once a week were 91.6 and 83.1, respectively. All treatment groups showed significant efficacy when compared to the vehicle control (P-values of <0.001). There was no statistical difference in the mycological efficacy between the treatment groups.

Conclusion.

Both CD101-treated groups showed significant clinical and mycological efficacy when compared to the vehicle control. There was no significant difference between treatment with CD101 35 mg/kg daily and animals treated with CD101 35 mg/kg once a week. Further evaluation of CD101 warranted.

TABLE 3
Summary of the clinical and mycological efficacy of
treatment groups as compared to the vehicle control.

| | Dose | Route of | Percent Efficacy | |
|---|---|---|---|---|
| Test Compounds | (mg/kg) | Administration | Mycological | Clinical |
| CD101 daily | 35 | SQ | 88.0* | 84.0* |
| CD101 once a week | 35 | SQ | 85.5* | 78.7* |
| Terbinafine daily | 10 | PO | 91.6* | 78.7* |
| Terbinafine once | 10 | PO | 83.1* | 60.6* |
| a week | | | | |
| Vehicle | NA | SQ | NA | NA |

*P-value of <0.001, when compared to the vehicle control.

### Example 2. Efficacy of Different Doses of CD101 in the Treatment of Trichophyton mentagrophytes Dermatophytosis in a Guinea Pig Model

In Example 1, we evaluated the efficacy of CD101 in treating dermatophytosis given subcutaneously at 35 mg/kg a day for 7 days, or once weekly. Example 1 demonstrated excellent efficacy when CD101 was used daily or once weekly. In the current example, we evaluated the efficacy of different doses of CD101 compared to terbinafine when animals were treated once weekly subcutaneously (SQ).

Procedures for animal care and use, preparation of standard inoculum, and infection of the animals were carried out following the same steps as described in Example 1.

Treatment Groups

Infected guinea pigs were randomized into the following (1)-(5) groups (10 per group) (see Table 4): (1) a vehicle control (water+mannitol); (2) terbinafine 10 mg/kg PO days 1 and 8, as a positive control; (3) CD101 10 mg/kg SQ days 1 and 8; (4) CD101 20 mg/kg SQ days 1 and 8; and (5) CD101 40 mg/kg SQ days 1 and 8.

TABLE 4
Schedule of Treatment

| Group | Test Agent | Administration | Dose |
|---|---|---|---|
| 1 | Vehicle (Water + Mannitol) | SubQ, day 1 and day 8 | NA |
| 2 | Terbinafine | PO, day 1 and day 8 | 10 mg/kg |
| 3 | CD101 (Water + Mannitol) | SubQ, day 1 and day 8 | 10 mg/kg |
| 4 | CD101 (Water + Mannitol) | SubQ, day 1 and day 8 | 20 mg/kg |
| 5 | CD101 (Water + Mannitol) | SubQ, day 1 and day 8 | 40 mg/kg |

Schedule of Treatment

Animals were treated with CD101 SQ or PO (for terbinafine) once a week on days 1 and 8.

a. Study Diagram (schedule)

b. Study Procedure

Study Day 1: Infect the animals with T. mentagrophytes and begin treatments.

Study Day 8: Last treatment.

Study Day 12: Clinical evaluation of the four quadrants in the marked area is done. Hair samples were obtained from each quadrant. Hairs (10 per quadrant) were planted in Potato Dextrose Agar, and incubated for 48 h for mycological evaluation. Procedures for clinical and mycological evaluations are described below.

c. Test Article Administration

The compounds was administered SQ or PO (0.3 mL/kg once daily).

Hair Root Invasion Test for Mycological Evaluation

Hair samples were removed with sterile forceps from four quadrants. Ten hair samples from each quadrant were inoculated onto the corresponding quadrant on the Potato Dextrose agar plate and incubated at 30° C. for 2 days. The fungal growth at the hair root was examined under a stereomicroscope. The effectiveness of a compound in reducing the number of mycologically positive hair sample per treated group was expressed as percentage relative to the untreated control group of animals using the following formula:

% Efficacy=100−(T×100/K)

where T=Positive hair in the test group, K=Positive hair in the Untreated control.

Clinical Evaluation

Clinical assessment of local changes of the infected skin area was scored as follows:

0=No lesions

1=Few slightly erythematous places on the skin

2=Well-defined redness, swelling with bristling hairs.

3=Large areas or marked redness incrustation, scaling, bald patches, ulcerated in places.

4=Partial damage to the integument and loss of hair

5=Extensive damage to the integument and complete loss of hair at the site of infection.

Any other local skin reactions were noted. The assessment of clinical evaluation in the change of scores per treatment group was expressed as percentage relative to the untreated control group of animals using the following formula:

% Efficacy=100−(T×100/K)

where T=Scores in the test group. K=Scores in the untreated control

Statistical Analysis

A statistical analysis of data from the clinical and mycological studies was performed. Significance was determined using test(s) as appropriate. The treated groups were compared to the untreated control to determine antifungal activity.

Results

Clinical Efficacy.

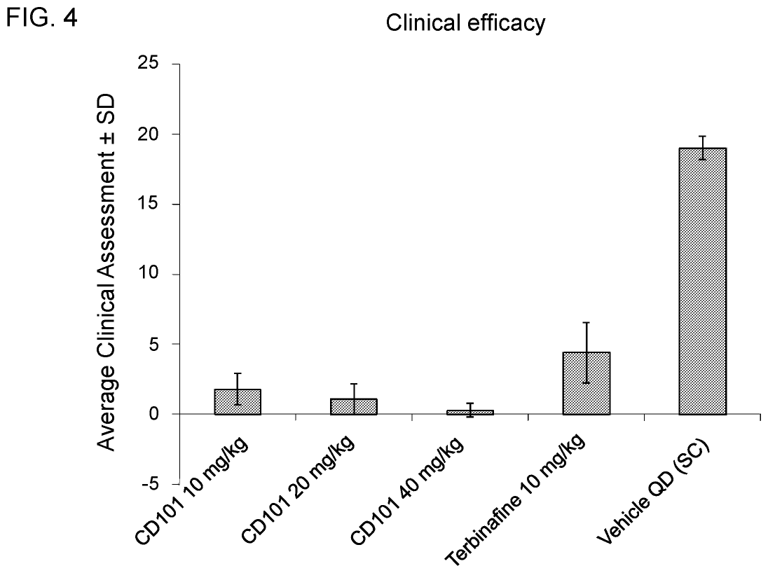

Table 5 and FIG. 4 show the clinical efficacy of each test compound as compared to the vehicle control. As expected, the vehicle control guinea pigs showed hair loss and ulcerated, scaly skin. Percent efficacies for CD101 10 mg/kg, CD101 20 mg/kg and CD101 40 mg/kg were 90.5, 94.2, and 98.4, respectively. The percent clinical efficacy for terbinafine 10 mg/kg was 76.8. All treatment groups showed significant efficacy when compared to the vehicle control (P-values of <0.001). The CD101-treated groups demonstrated significant efficacy when compared the terbinafine-treated group (P-values of <0.001). There was no significant difference between the CD101-treated groups.

Mycological Efficacy.

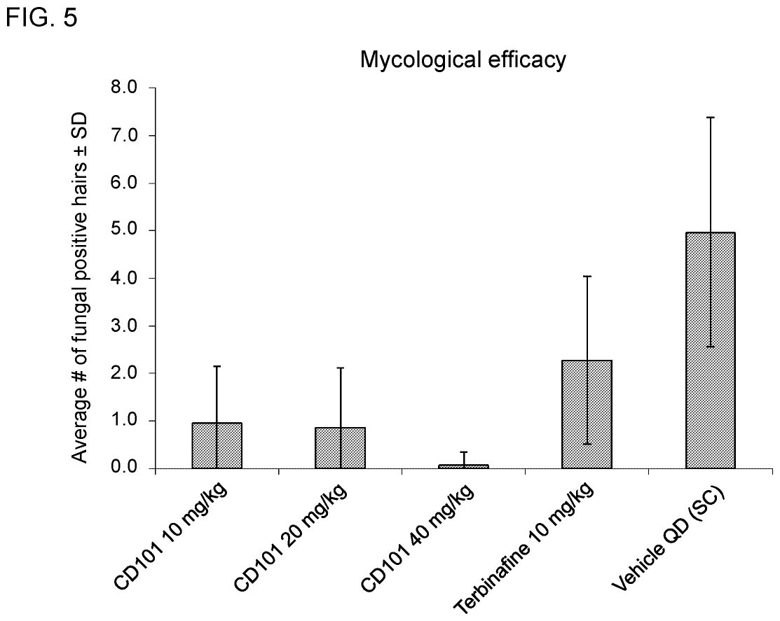

Table 5 and FIG. 5 show the mycological efficacy of each test compound as compared to the vehicle control. The vehicle control behaved as expected having the highest average fungal positive hairs. Percent clinical efficacies for CD101 10 mg/kg, CD101 20 mg/kg and CD101 40 mg/kg were 80.9, 82.9, and 98.5%, respectively, while the percent clinical efficacy for terbinafine 10 mg/kg was 54.2%. All treatment groups showed significant efficacy when compared to the vehicle control (P-values of <0.001). The CD101-treated groups demonstrated significant efficacy when compared with the terbinafine-treated group (P-values of 0.001). There was no significant difference between the CD101-treated groups. Comparing the mycological efficacy of CD101 40 mg/kg-treated group to the CD101 20 mg/kg and 10 mg/kg-treated groups showed a trend towards dose dependent efficacy (P-values of 0.220 and 0.098, respectively).

Conclusion.

All CD101-treated groups showed significant clinical and mycological efficacy when compared to the vehicle control, as well as to the terbinafine-treated group. CD101 demonstrated a trend towards a greater mycological efficacy compared to terbinafine at higher doses. Our findings demonstrate that CD101 possesses potent mycological and clinically efficacy when dosed once weekly. Further evaluation s warranted.

TABLE 5
Summary of the clinical and mycological efficacy
of once a week treatment (days 1 and 8) groups
as compared to the vehicle control.

| | Dose | Route of | Percent Efficacy | |
|---|---|---|---|---|
| Test Compounds | (mg/kg) | Administration | Mycological | Clinical |
| CD101 | 10 | SQ | 80.9* | 90.5* |
| CD101 | 20 | SQ | 82.9* | 94.2* |
| CD101 | 40 | SQ | 98.5* | 98.4* |
| Terbinafine | 10 | PO | 54.2* | 76.8* |
| Vehicle QD (SQ) | NA | SQ | NA | NA |

*P-value of <0.001, when compared to the vehicle control.

### Example 3. Subcutaneous Injection of CD101 in Cynomolgus Monkeys

In this study, the tissue concentration of subcutaneously injected CD101 was evaluated in cynomolgus monkeys.

Study Design

CD101 in the vehicle (1.14% mannitol in sterile water for injection, pH 6.0) was administered by subcutaneous injection once every 3 days (5 doses) over a 2-week period to 3 groups (Groups 2, 3, and 4) of cynomolgus monkeys. Dose levels were 30, 60, and 120 mg/kg/dose for Groups 2, 3, and 4, respectively. A concurrent control group (Group 1) received the vehicle on a comparable regimen. The dose volumes were 1.2, 0.3, 0.6, and 1.2 mL/kg spread evenly over 4, 1, 2, and 4 dose sites for Groups 1-4, respectively, on each day of dosing. The control and 120 mg/kg/dose groups each consisted of 5 males and 5 females, and the 30 and 60 mg/kg/dose groups each consisted of 3 males and 3 females.

The animals were observed twice daily for mortality and moribundity. Clinical examinations were performed once daily for at least 6 consecutive days, including the day of randomization, prior to randomization, prior to dosing, 1-2 hours postdosing, once daily on nondosing days, and once daily during the recovery period. Detailed physical examinations were performed at least weekly during the acclimation period until randomization, on the day of randomization, weekly (±2 days) during the study period, and on the day of the scheduled necropsies. Injection site (IS) observations were performed prior to dosing (dose sites that were utilized on that day of dosing), 1-2 hours postdosing (dose sites that were utilized on that day of dosing), once daily on nondosing days (dose sites that had been previously been utilized), and twice weekly during the recovery period. Individual body weights were recorded weekly (±2 days). Individual body weights were collected at least weekly during the acclimation period until randomization, on the day of randomization, Study Day 0 (prior to dosing), weekly (2±days) during the study period, on the day prior to the day of the scheduled necropsies (nonfasted) and on the day of the scheduled necropsies (fasted). Clinical pathology parameters (hematology, coagulation, serum chemistry, and urinalysis) were analyzed during acclimation prior to randomization (Study Day −15), and on the day of the primary (Study Day 17) and recovery (Study Day 45) necropsies. Blood samples for toxicokinetic evaluation were collected from all animals/sex/group/time point prior to dose administration and at approximately 1, 2, 4, 8, 24, 48, and 72 hours after dose administration on Study Days 0 and 12. Complete necropsies were performed on all animals, and selected organs were weighed at the scheduled necropsies. Selected tissues were examined microscopically from all animals. Fingernail, toenail, nail bed, and abdominal skin samples were collected from all animals at the scheduled necropsies for bioanalytical analysis.

Results
All animals survived to the scheduled necropsies. There were no test article-related clinical observations or effects on body weights.

On Study Day 0, concentrations of CD101 reached maximum or near maximum observed values by 8 hours postdose and concentrations remained approximately constant through 72 hours. On Study Day 12, CD101 concentrations at predose were approximately 2-fold greater than Study Day 0 Cₘₐₓ concentrations and remained approximately constant through 72 hours. There was no notable difference in exposure (AUC0-72 and Cₘₐₓ) of CD101 between male and female Cynomolgus monkeys and combined sex parameters are reported. Overall, combined exposure (AUC0-72 and Cₘₐₓ) increased in a dose-proportional manner on Study Day 0 and in a slightly lower than dose-proportional (approximately 3-fold over a 4-fold dose change) manner on Study Day 12 across all dose ranges. There was notable plasma accumulation at all dosage levels in all monkeys with combined accumulation ratios of 3.18, 2.70, and 2.67 at the 30, 60, and 120 mg/kg/dose dosage levels, respectively. Cₘₐₓ values range from 21.0 to 65.1 μg/mL and AUC0-72 ranged from 1365 to 4315 μg·h/mL across the 30 to 120 mg/kg groups (males and females combined). There was dose-dependent increases in tissue levels of CD101 in the fingernail, fingernail bed, toenail, toenail bed, and skin with no apparent difference in the tissue exposures between male and female animals. Mean values ranged from 40.4 to 350 μg/g across the 30 to 120 mg/kg dose groups.

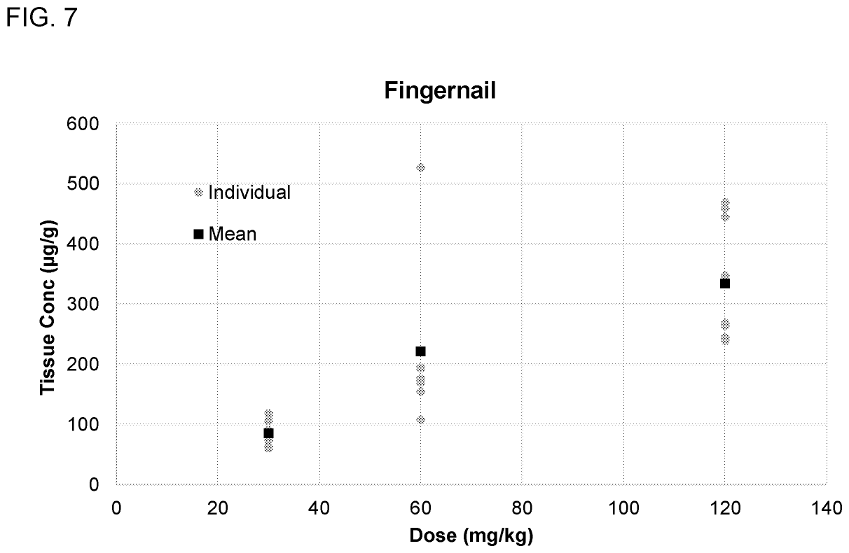

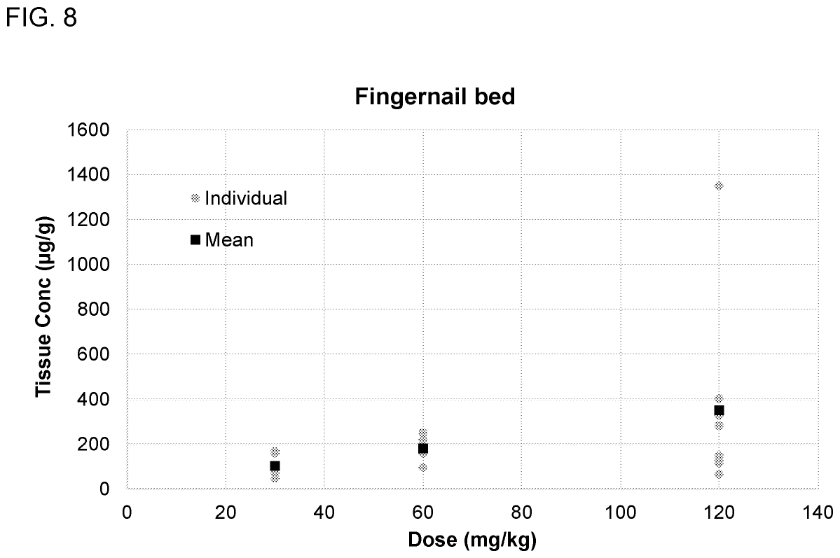

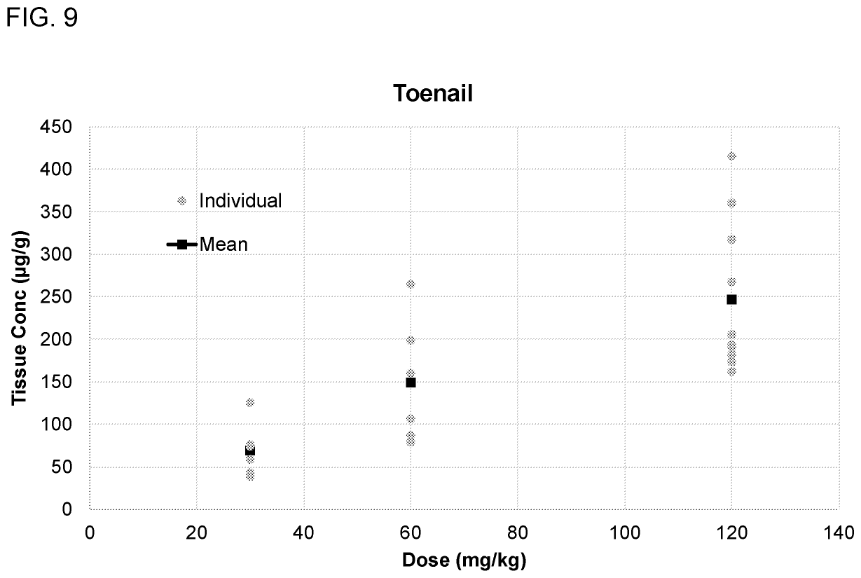

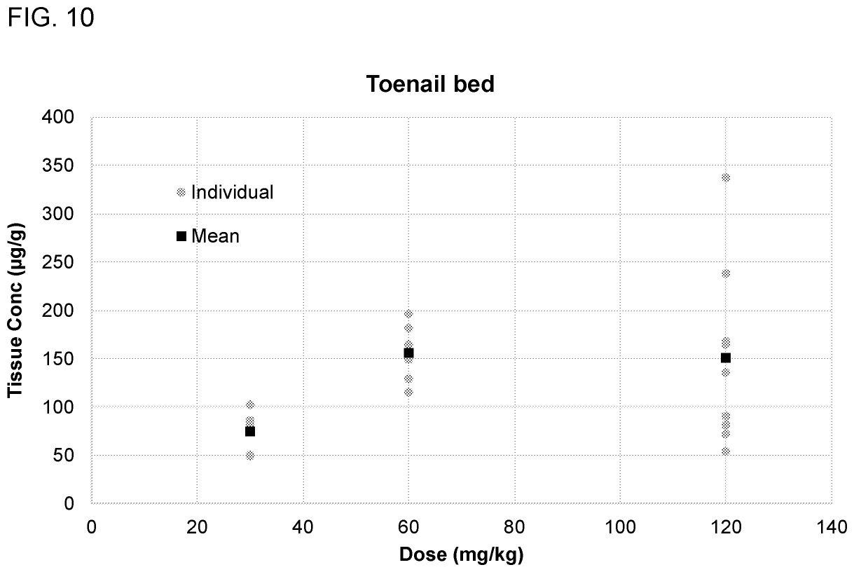

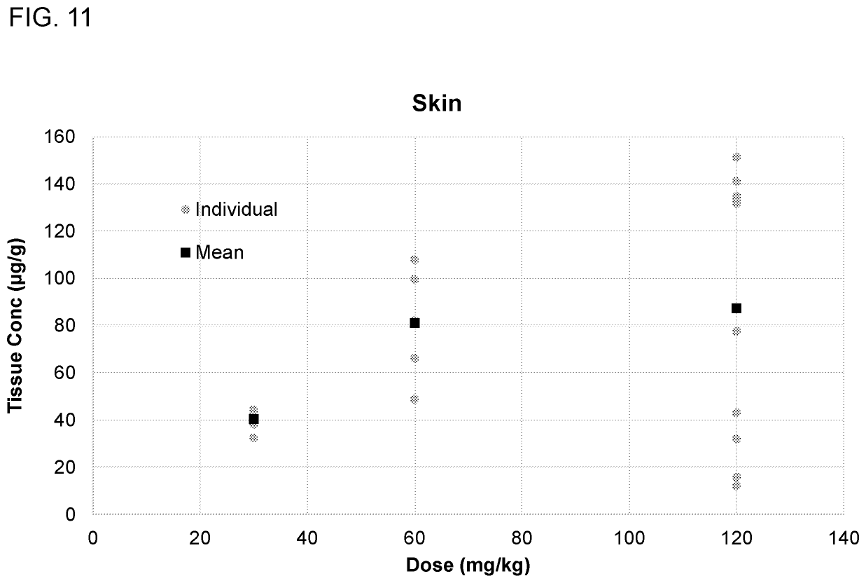

As there was no apparent difference in the tissue exposures between male and female animals, the concentrations were sorted by matrix/dose and averaged across the different doses tested (Table 6). Graphs (in FIGS. 7-11) were plotted based on individual values as well as mean concentrations. Although considerable inter-animal variability was observed, in general, the tested tissues (fingernail, fingernail bed, toenail, toenail bed, and skin) show dose-dependent increases in exposures. Mean fingernail concentrations ranged from 85.5 to 334 μg/g (FIG. 7) while mean fingernail bed concentrations ranged from 103 to 350 μg/g (FIG. 8) across the 30 to 120 mg/kg doses. Mean toenail concentrations ranged from 69.3 to 247 μg/g (FIG. 9) while mean toenail bed concentrations ranged from 74.7 to 151 μg/g (FIG. 10) across the 30 to 120 mg/kg doses. Mean skin concentrations ranged from 40.4 to 87.3 μg/g (FIG. 11) across the 30 to 120 mg/kg doses.

### CONCLUSIONS

These results suggest that subcutaneously injected CD101 reached appreciable tissue concentrations in the ug/g level in cynomolgus monkeys. Exposures from a dose of 10-20 mg/kg CD101 in monkeys should be comparable to an intravenous dose of 400 mg CD101 in humans.

TABLE 6
Summary of CD101 concentration in different tissues following
subcutaneous injection of CD101 in cynomolgus monkeys.

| | | | | | CD101 | Mean |
|---|---|---|---|---|---|---|
| | | | | | Concen- | Concen- |
| | | | Dose | | tration | tration |
| Animal | Sex | Group | (mg/kg) | Matrix | (μg/g) | (μg/g) |
| 1499 | Male | 2 | 30 | Fingernail | 90.0 | 85.5 |
| 1503 | Male | 2 | 30 | Fingernail | 106 | |
| 1513 | Male | 2 | 30 | Fingernail | 118 | |
| 1516 | Female | 2 | 30 | Fingernail | 63.6 | |
| 1529 | Female | 2 | 30 | Fingernail | 60.8 | |
| 1531 | Female | 2 | 30 | Fingernail | 74.2 | |
| 1498 | Male | 3 | 60 | Fingernail | 194 | 222 |
| 1508 | Male | 3 | 60 | Fingernail | 108 | |
| 1512 | Male | 3 | 60 | Fingernail | 527 | |
| 1522 | Female | 3 | 60 | Fingernail | 171 | |
| 1524 | Female | 3 | 60 | Fingernail | 155 | |
| 1525 | Female | 3 | 60 | Fingernail | 175 | |
| 1501 | Male | 4 | 120 | Fingernail | 266 | 334 |
| 1504 | Male | 4 | 120 | Fingernail | 264 | |
| 1506 | Male | 4 | 120 | Fingernail | 445 | |
| 1507 | Male | 4 | 120 | Fingernail | 469 | |
| 1509 | Male | 4 | 120 | Fingernail | 239 | |
| 1521 | Female | 4 | 120 | Fingernail | 244 | |
| 1523 | Female | 4 | 120 | Fingernail | 269 | |
| 1526 | Female | 4 | 120 | Fingernail | 459 | |
| 1532 | Female | 4 | 120 | Fingernail | 348 | |
| 1533 | Female | 4 | 120 | Fingernail | 339 | |
| 1499 | Male | 2 | 30 | Fingernail bed | 168 | 103 |
| 1503 | Male | 2 | 30 | Fingernail bed | 160 | |
| 1513 | Male | 2 | 30 | Fingernail bed | 92.5 | |
| 1516 | Female | 2 | 30 | Fingernail bed | 75.9 | |
| 1529 | Female | 2 | 30 | Fingernail bed | 71.9 | |
| 1531 | Female | 2 | 30 | Fingernail bed | 48.6 | |
| 1498 | Male | 3 | 60 | Fingernail bed | 218 | 181 |
| 1508 | Male | 3 | 60 | Fingernail bed | 96.7 | |
| 1512 | Male | 3 | 60 | Fingernail bed | 251 | |
| 1522 | Female | 3 | 60 | Fingernail bed | 159 | |
| 1524 | Female | 3 | 60 | Fingernail bed | 175 | |
| 1525 | Female | 3 | 60 | Fingernail bed | 184 | |
| 1501 | Male | 4 | 120 | Fingernail bed | 1351 | 350 |
| 1504 | Male | 4 | 120 | Fingernail bed | 404 | |
| 1506 | Male | 4 | 120 | Fingernail bed | 67.1 | |
| 1507 | Male | 4 | 120 | Fingernail bed | 129 | |
| 1509 | Male | 4 | 120 | Fingernail bed | 346 | |
| 1521 | Female | 4 | 120 | Fingernail bed | 332 | |
| 1523 | Female | 4 | 120 | Fingernail bed | 330 | |
| 1526 | Female | 4 | 120 | Fingernail bed | 147 | |
| 1532 | Female | 4 | 120 | Fingernail bed | 283 | |
| 1533 | Female | 4 | 120 | Fingernail bed | 114 | |
| 1499 | Male | 2 | 30 | Toenail | 76.0 | 69.3 |
| 1503 | Male | 2 | 30 | Toenail | 74.2 | |
| 1513 | Male | 2 | 30 | Toenail | 126 | |
| 1516 | Female | 2 | 30 | Toenail | 58.8 | |
| 1529 | Female | 2 | 30 | Toenail | 42.8 | |
| 1531 | Female | 2 | 30 | Toenail | 38.3 | |
| 1498 | Male | 3 | 60 | Toenail | 265 | 149 |
| 1508 | Male | 3 | 60 | Toenail | 86.7 | |
| 1512 | Male | 3 | 60 | Toenail | 198 | |
| 1522 | Female | 3 | 60 | Toenail | 106 | |
| 1524 | Female | 3 | 60 | Toenail | 160 | |
| 1525 | Female | 3 | 60 | Toenail | 79.2 | |
| 1501 | Male | 4 | 120 | Toenail | 268 | 247 |
| 1504 | Male | 4 | 120 | Toenail | 182 | |
| 1506 | Male | 4 | 120 | Toenail | 360 | |
| 1507 | Male | 4 | 120 | Toenail | 162 | |
| 1509 | Male | 4 | 120 | Toenail | 416 | |
| 1521 | Female | 4 | 120 | Toenail | 173 | |
| 1523 | Female | 4 | 120 | Toenail | 206 | |
| 1526 | Female | 4 | 120 | Toenail | 318 | |
| 1532 | Female | 4 | 120 | Toenail | 190 | |
| 1533 | Female | 4 | 120 | Toenail | 193 | |
| 1499 | Male | 2 | 30 | Toenail bed | 76.9 | 74.7 |
| 1503 | Male | 2 | 30 | Toenail bed | 102 | |
| 1513 | Male | 2 | 30 | Toenail bed | 50.5 | |
| 1516 | Female | 2 | 30 | Toenail bed | 86.1 | |
| 1529 | Female | 2 | 30 | Toenail bed | 82.9 | |
| 1531 | Female | 2 | 30 | Toenail bed | 49.3 | |
| 1498 | Male | 3 | 60 | Toenail bed | 196 | 156 |
| 1508 | Male | 3 | 60 | Toenail bed | 116 | |
| 1512 | Male | 3 | 60 | Toenail bed | 182 | |
| 1522 | Female | 3 | 60 | Toenail bed | 129 | |
| 1524 | Female | 3 | 60 | Toenail bed | 165 | |
| 1525 | Female | 3 | 60 | Toenail bed | 149 | |
| 1501 | Male | 4 | 120 | Toenail bed | 166 | 151 |
| 1504 | Male | 4 | 120 | Toenail bed | 337 | |
| 1506 | Male | 4 | 120 | Toenail bed | 72.2 | |
| 1507 | Male | 4 | 120 | Toenail bed | 54.1 | |
| 1509 | Male | 4 | 120 | Toenail bed | 238 | |
| 1521 | Female | 4 | 120 | Toenail bed | 136 | |
| 1523 | Female | 4 | 120 | Toenail bed | 168 | |
| 1526 | Female | 4 | 120 | Toenail bed | 90.6 | |
| 1532 | Female | 4 | 120 | Toenail bed | 165 | |
| 1533 | Female | 4 | 120 | Toenail bed | 81.6 | |
| 1499 | Male | 2 | 30 | Skin | 40.9 | 40.4 |
| 1503 | Male | 2 | 30 | Skin | 32.4 | |
| 1513 | Male | 2 | 30 | Skin | 42.8 | |
| 1516 | Female | 2 | 30 | Skin | 38.2 | |
| 1529 | Female | 2 | 30 | Skin | 44.4 | |
| 1531 | Female | 2 | 30 | Skin | 43.7 | |
| 1498 | Male | 3 | 60 | Skin | 108 | 81.0 |
| 1508 | Male | 3 | 60 | Skin | 66.2 | |
| 1512 | Male | 3 | 60 | Skin | 99.6 | |
| 1522 | Female | 3 | 60 | Skin | 48.8 | |
| 1524 | Female | 3 | 60 | Skin | 81.7 | |
| 1525 | Female | 3 | 60 | Skin | 82.0 | |
| 1501 | Male | 4 | 120 | Skin | 133 | 87.3 |
| 1504 | Male | 4 | 120 | Skin | 132 | |
| 1506 | Male | 4 | 120 | Skin | 32.0 | |
| 1507 | Male | 4 | 120 | Skin | 12.2 | |
| 1509 | Male | 4 | 120 | Skin | 141 | |
| 1521 | Female | 4 | 120 | Skin | 151 | |
| 1523 | Female | 4 | 120 | Skin | 77.6 | |
| 1526 | Female | 4 | 120 | Skin | 43.1 | |
| 1532 | Female | 4 | 120 | Skin | 135 | |
| 1533 | Female | 4 | 120 | Skin | 15.9 | |

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. A method of treating onychomycosis in a subject, the method comprising subcutaneously administering to the subject doses of about 25 mg to about 600 mg of CD101, 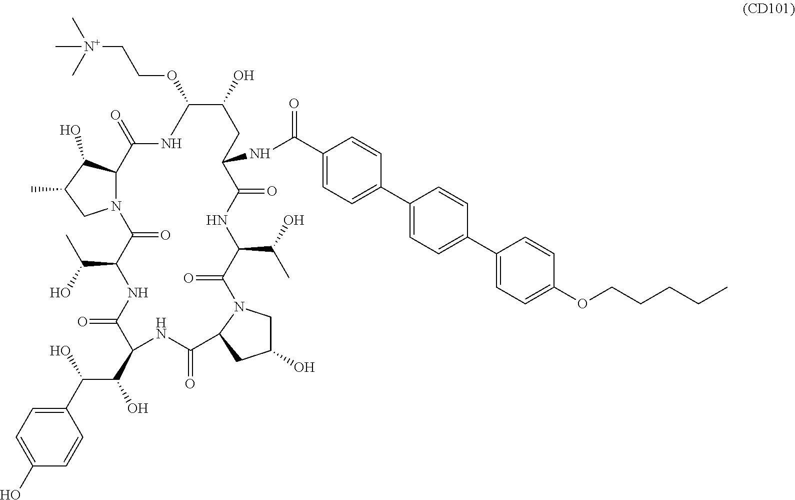 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1]
in salt or neutral form, wherein the CD101 in salt or neutral form is administered in one or more doses to the subject over a period of 4 to 52 weeks and wherein no more than one dose is administered per week.

2. The method of claim 1, wherein one dose of CD101 in salt or neutral form is administered once every 4 weeks over a period of 4 to 52 weeks.

3. The method of claim 1, wherein one dose of CD101 in salt or neutral form is administered once every 3 weeks over a period of 6 to 51 weeks.

4. The method of claim 1, wherein one dose of CD101 in salt or neutral form is administered once every 2 weeks over a period of 4 to 52 weeks.

5. The method of claim 1, wherein one dose of CD101 in salt or neutral form is administered once a weeks over a period of 4 to 52 weeks.

6. The method of any one of claims 1-5, wherein one or more doses of CD101 is administered over a period of 12 to 24 weeks.

7. The method of claim 6, wherein 3 to 12 doses of CD101 is administered over a period of 12 weeks.

8. The method of claim 6, wherein 4 to 16 doses of CD101 is administered over a period of 16 weeks.

9. The method of claim 6, wherein 5 to 20 doses of CD101 is administered over a period of 20 weeks.

10. The method of claim 6, wherein 6 to 24 doses of CD101 is administered over a period of 24 weeks.

11. The method of any one of claims 1-10, wherein the subject has failed treatment with an oral therapy for onychomycosis.

12. The method of claim 11, wherein the subject has failed treatment with terbinafine, itraconazole, fluconazole, posaconazole, and/or griseofulvin.

13. The method of any one of claims 1-12, wherein the subject has failed treatment with a topical therapy for onychomycosis.

14. The method of claim 13, wherein the subject has failed treatment with ketoconazole, miconazole, butenafine, and/or naftifine.

15. The method of any one of claims 1-14, wherein the subject has failed treatment with a nonpharmacological therapy for onychomycosis.

16. The method of claim 15, wherein the subject as failed mechanical, chemical, or surgical nail avulsion, laser treatment, and/or photodynamic treatment.

17. The method of any one of claims 1-16, further comprising administering to the subject at least one antifungal agent.

18. The method of claim 17, wherein CD101 in salt or neutral form and the antifungal agent are administered substantially simultaneously.

19. The method of claim 17, wherein CD101 in salt or neutral form and the antifungal agent are administered separately.

20. The method of claim 19, wherein CD101 in salt or neutral form is administered first, followed by administration of the antifungal agent.

21. The method of claim 19, wherein the antifungal agent is administered first, followed by administration of CD101 in salt or neutral form.

22. The method of claim 17, wherein CD101 in salt or neutral form and the antifungal agent are administered substantially simultaneously, followed by administration of CD101 in salt or neutral form or the antifungal agent alone.

23. The method of claim 17, wherein CD101 in salt or neutral form or the antifungal agent is administered alone, followed by administering of CD101 in salt or neutral form and the antifungal agent substantially simultaneously.

24. The method of any one of claims 17-23, wherein the antifungal agent is an allylamine compound, an azole compound, an echinocandin compound, a polygene compound, flucytosine (Ancobon®)), enfumafungin, or SCY-078.

25. The method of any one of claims 17-24, wherein the antifungal agent is terbinafine, itraconazole, fluconazole, posaconazole, griseofulvin, ketoconazole, miconazole, butenafine, or naftifine.

26. A method of preventing or reducing the likelihood of onychomycosis in a subject, the method comprising subcutaneously administering to the subject CD101 in salt or neutral form.

27. The method of claim 26. wherein the subject is in a population with high prevalence of onychomycosis.

28. The method of claim 26 or 27, wherein the population is a population of soldiers, long-distance runners, or miners.

29. The method of any one of claims 1-28, wherein the onychomycosis is a dermatophytic onychomycosis.

30. The method of claim 29, wherein the dermatophytic onychomycosis is caused by a fungus in the genus Trichophyton, Microsporum, or Epidermophyton.

31. The method of claim 30, wherein the fungus is in the genus Trichophyton.

32. The method of claim 31, wherein the fungus is Trichophyton rubrum, T. mentagrophytes, T. interdigitale, T. violaceum, T. tonsurans, T. soudanense, T. concentricum, T. megnini, T. schoenleinii, T. yaoundei, T. verrucosum, T. simii, T. redellii, T. equinum, T. ajelloi, T. vanbreuseghemii, T. terrestre, T. phaseoliforme, T. flavescens, T. gloriae, or T. onychocola.

33. The method of claim 32, wherein the fungus is T. rubrum or T. mentagrophytes.

34. The method of claim 30, wherein the fungus is in the genus Microsporum.

35. The method of claim 34, wherein the fungus is Microsporum gypseum, M. amazonicum, M. audouinii, M. boullardii, M. canis, M. canis, M. cookei, M. distortum, M. duboisii, M. equinum, M. ferrugineum, M. fulvum, M. gallinae, M. langeronii, M. nanum, M. persicolor, M. praecox, M. ripariae, or M. rivalieri.

36. The method of claim 30, wherein the fungus is in the genus Epidermophyton.

37. The method of claim 36, wherein the fungus is Epidermophyton floccosum or E. stockdaleae.

38. The method of any one of claims 1-28, wherein the onychomycosis is a non-dermatophytic onychomycosis.

39. The method of claim 38, wherein the non-dermatophytic onychomycosis is caused by Candida albicans, C. glabrata, C. dubliniensis, C. krusei, C. parapsilosis, C. tropicalis, C. orthopsilosis, C. guilliermondii, C. rugosa, or C. lusitaniae.

40. The method of claim 39, where the non-dermatophytic onychomycosis is caused by Candida albicans.

41. The method of any one of claims 1-40, wherein the onychomycosis is in the nail plate, nail matrix, nail bed, nail cuticle, nail lunula, nail root, nail sinus, nail hyponychium, nail free margin, or any combination thereof.

42. The method of any one of claims 1-41, wherein the onychomycosis is distal subungual onychomycosis, white superficial onychomycosis, proximal subungual onychomycosis, candidal onychomycosis, or total dystrophic onychomycosis.

43. The method of any one of claims 1-42, wherein the subcutaneous administration of CD101 in salt or neutral form does not cause any negative injection site effects.

44. The method of any one of claims 1-43, wherein CD101 is administered as an aqueous pharmaceutical composition.

45. The method of claim 44, wherein the pharmaceutical composition has a pH of from 4 to 8.

46. The method of any one of claims 1-45, wherein CD101 in salt form is CD101 acetate.
